(19) United States
(12) Patent Application Publication
(10) Pub. No.: US 2018/0057530 A1
(43) Pub. Date: Mar. 1, 2018

(54) CYCLIC PEPTIDE COMPOUND, AND PREPARATION AND USE THEREOF

(71) Applicant: Zhejiang Hisun Pharmaceutical Co., Ltd., Taizhou (CN)

(72) Inventors: Hua Bai, Taizhou (CN); Niefang Yu, Taizhou (CN); Jidong Wang, Taizhou (CN); Xiaohe Zheng, Taizhou (CN); Lifei Liu, Taizhou (CN); Jiansheng Huang, Taizhou (CN)

(73) Assignee: Zhejiang Hisun Pharmaceutical Co., Ltd., Taizhou (CN)

(21) Appl. No.: 15/559,283

(22) PCT Filed: Apr. 1, 2016
(86) PCT No.: PCT/CN2016/078303

§ 371 Date: Sep. 18, 2017

(30) Foreign Application Priority Data
Apr. 3, 2015 (CN) 201510158661.1

(51) Int. Cl. C07K 5/103

(52) U.S. Cl. CPC C07K 5/101; A61K 38/00

(57) ABSTRACT

The present invention relates to a compound represented by formula (I). The present invention also relates to the use of a compound represented by formula (I) in the treatment of disorders resulting from cell proliferation and/or angiogenesis or disorders associated with or accompanying cell proliferation and/or angiogenesis.

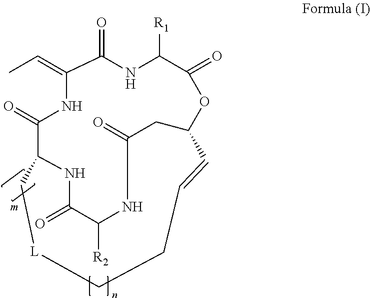

### TECHNICAL FIELD

The present invention relates to novel cyclic peptide derivatives, more particularly, to the acquisition of novel derivatives by converting a specific sulfur group on a cyclic peptide into a sulfone or sulfoxide structure. In addition, the present invention relates to a medicament containing these compounds and to the use of these compounds in the manufacture of a medicament.

### BACKGROUND ART

Cyclic peptide compounds are a very important class of drug basic structures. In cyclic peptide compounds, sulfur-containing or sulfur bridge-containing cyclic peptide derivatives are very characteristic, for example, Romidepsin.

In 1994, researchers from Fujisawa first reported isolation of Romidepsin from Chromobacterium violaceum. This compound, having only very weak or no antibacterial activity, inhibits various tumor cells without affecting normal cells. Animal tumor model tests showed that Romidepsin has a significant anticancer effect (Ueda H, Nakajima H, Hori Y, et al. FR901228, a novel antitumor bicyclic depsipeptide produced by Chromobacterium violaceum No. 968. I. Taxonomy, fermentation, isolation, physico-chemical and biological properties, and antitumor activity[J]. The Journal of antibiotics, 1994, 47(3): 301-310.).

In 1996, researchers at Harvard University first reported total synthesis of Romidepsin (Li K W, Wu J, Xing W, et al. Total synthesis of the antitumor depsipeptide FR-901,228[J]. Journal of the American Chemical Society, 1996, 118(30): 7237-7238.). However, its mechanism of action was not confirmed until 1998, when it was comprehensively compared with trichostatin A, an HDAC inhibitor (Nakajima H, Kim Y B, Terano H, et al. FR901228, a potent antitumor antibiotic, is a novel histone deacetylase inhibitor[J]. Experimental cell research, 1998, 241(1): 126-133.).

Funded by the US National Cancer Institute (NCI), in 1997, a clinical phase I study was performed on Romidepsin. In a later phase II clinical trial, treatment experiment research was conducted on multiple myeloma, prostate cancer, breast cancer, ovarian cancer, lung cancer, etc. However, the best therapeutic effect was obtained from the treatment studies on cutaneous T-cell lymphoma and peripheral T-cell lymphoma (Clinical Trials.gov NCT00098397. Retrieved on Nov. 8, 2009. Clinical Trials.gov NCT00085527. Retrieved on Nov. 8, 2009. Clinical Trials.gov NCT00104884. Retrieved on Nov. 8, 2009. Clinical Trials.gov NCT00084461. Retrieved on Nov. 8, 2009. Clinical Trials.gov NCT00062075. Retrieved on Nov. 8, 2009).

In 2004, Romidepsin received an FDA approval and entered the “fast track,” intended for the treatment of cutaneous T-cell lymphoma. In addition, the FDA and the European Medicines Evaluation Agency (EMEA) approved the use of Romidepsin in the development of orphan drugs, for the treatment of certain special tumors. Finally, Romidepsin was approved by the FDA in 2009 for marketing for the treatment of cutaneous T-cell lymphoma.

However, as Romidepsin has certain side effects, including anemia, thrombocytopenia, and leucopenia, etc, a large number of structural optimization efforts were attracted to improve its efficacy while further reducing its side effects ([No authors listed] (November 2009). “ISTODEX Label Information”. U.S. Food and Drug Administration. Retrieved 2009-11-07).

### SUMMARY OF THE INVENTION

The present invention transforms the sulphur, particularly sulfur bridge atoms, on cyclic peptide molecules into a structure containing sulfone or sulfoxide, thereby obtaining a new class of derivatives, with a general chemical formula shown in formula (I):

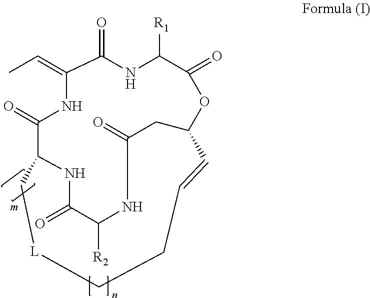

wherein, n=0, 1, 2; m=0, 1.

R₁ and R₂ are independently selected from H, alkyl, nitro, cyano, halo, haloalkyl, haloalkenyl, hydroxy, hydroxyalkyl, alkoxy, alkoxycarbonyl, aryloxy, alkenyloxy, alkynyloxy, cycloalkoxy, heterocycloalkoxy, amino, alkylamino, aminoalkyl, amide group, alkylaminocarbonyl, sulfonyl, alkylsulfonyl, alkylsulfinyl, aminosulfonyl, acyl, cycloalkyl, cycloalkenyl, heterocycloalkyl, aryl, heteroaryl, cycloalkylalkyl, arylalkyl, heteroarylalkyl, cycloalkylheteroalkyl, heterocycloalkylheteroalkyl, heteroarylheteroalkyl, or arylheteroalkyl.

L is selected from:

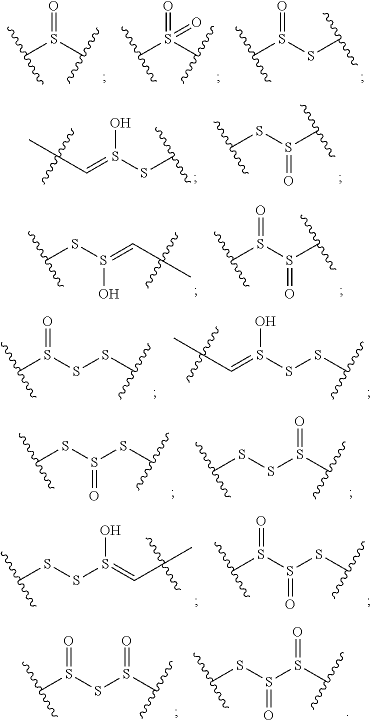

In a preferred embodiment of the present invention, R₁ and R₂ are independently preferably selected from H, C₁-C₆ alkyl or C₁-C₆ alkoxy.

In a preferred embodiment of the present invention, L is preferably selected from:

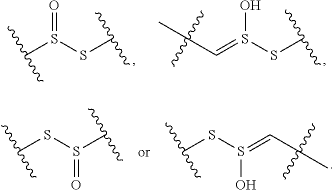 [SMILES: *SS(*)=O ; */C=S(\O)S* ; *SS(*)=O ; */C=S(\O)S*]

In a preferred embodiment of the present invention, the compound of formula (I) is selected from:

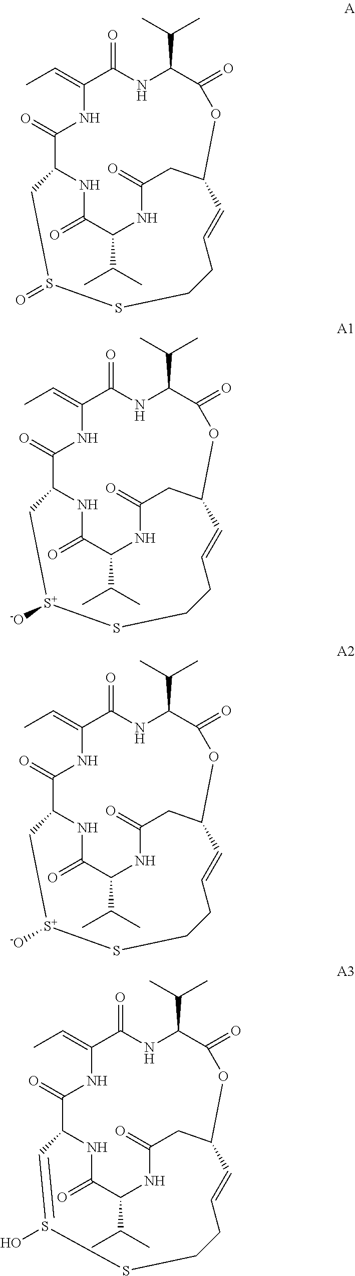

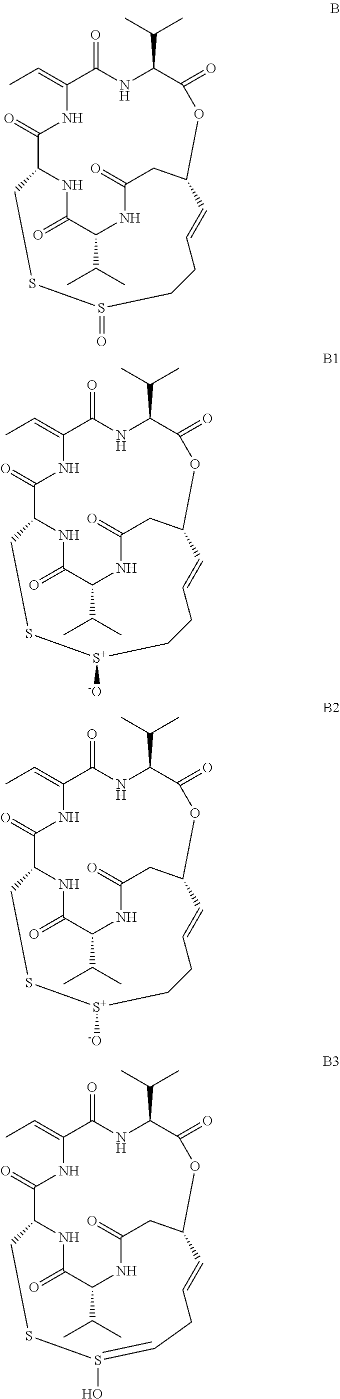

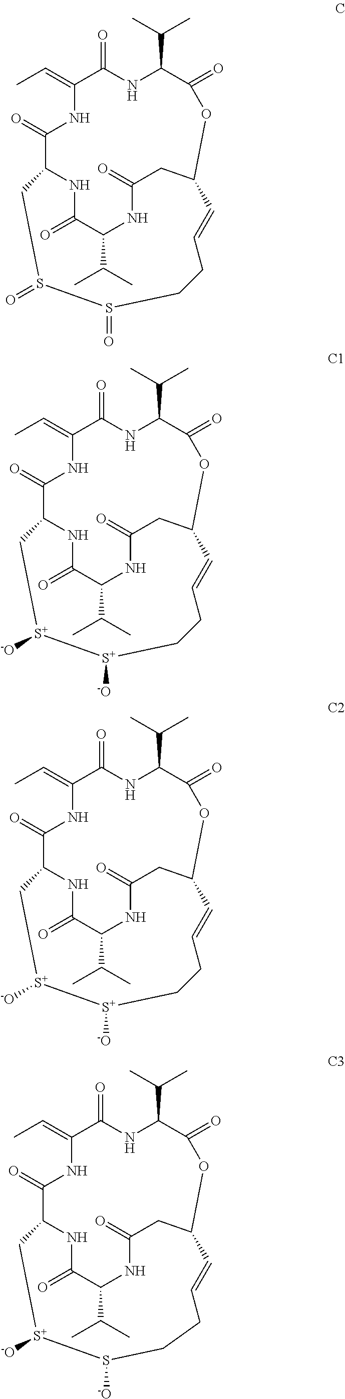

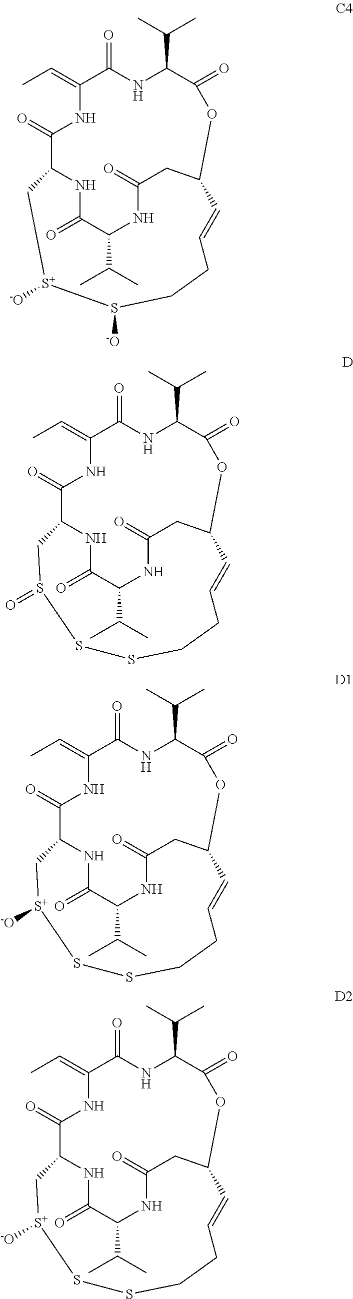

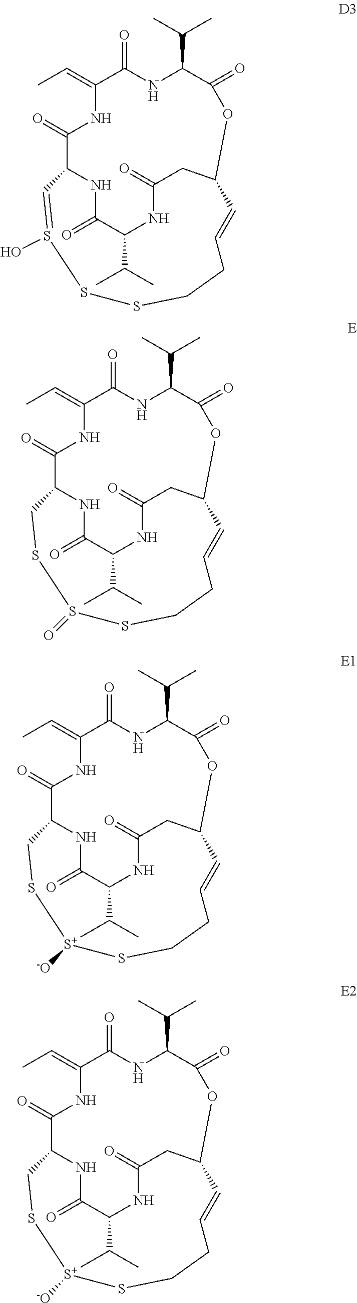

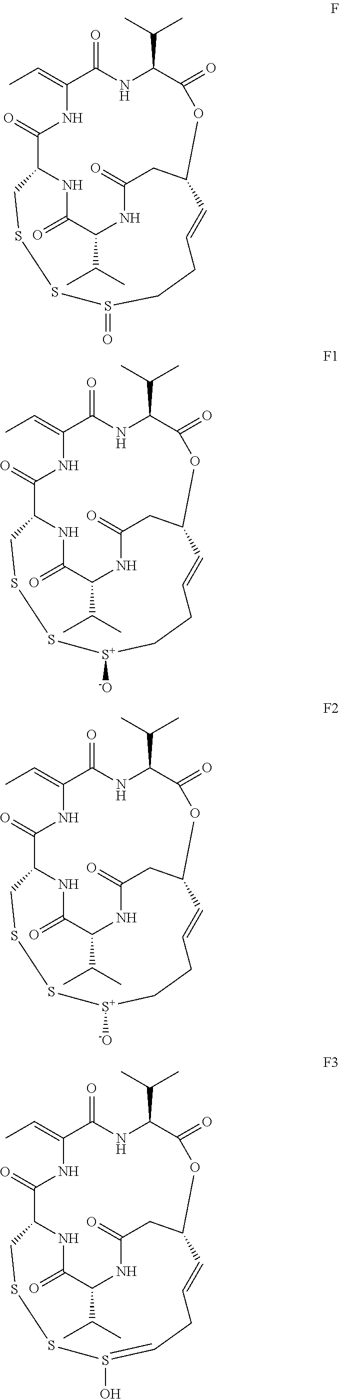

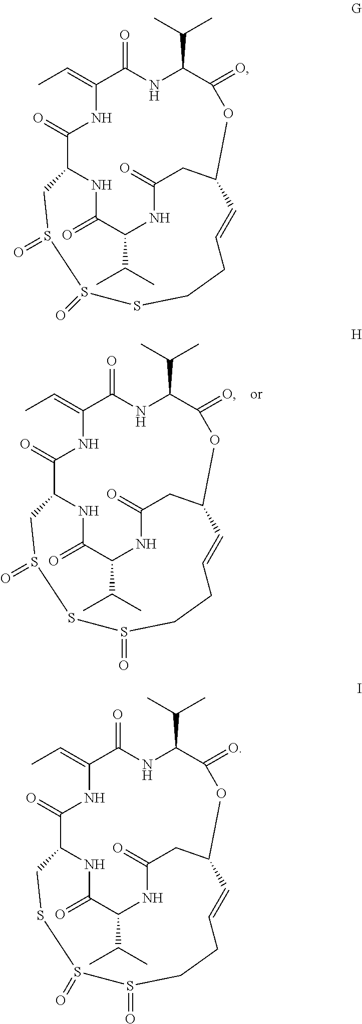

The present invention provides a method for preparing a compound of formula (I) or a pharmaceutically acceptable salt, stereoisomer or an optical isomer, said method comprising: oxidizing the compound of formula (II) with an oxidant to obtain the compound of formula (I)

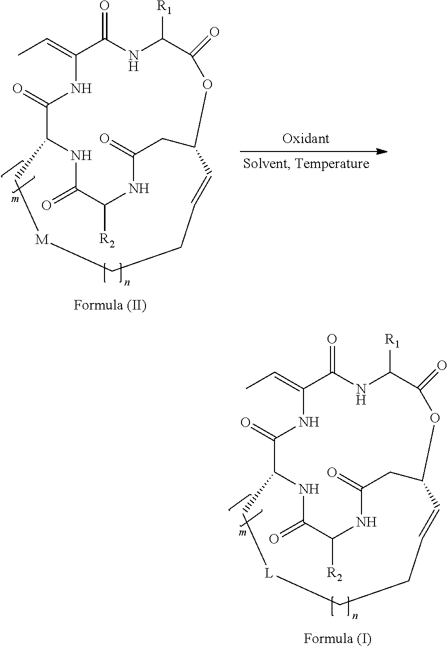

wherein, R₁, R₂, L, n and m are as defined above; M is selected from —S—, —SS—, or —SSS—.

In a preferred embodiment, the oxidant is selected from any one or more of m-chloroperbenzoic acid, peracetic acid, potassium peroxymonosulfate (Oxone), cumene hydroperoxide, tert-butyl hydroperoxide, sodium periodate, acetone peroxide and hydrogen peroxide (H₂O₂).

In a preferred embodiment, the method comprises: dissolving the starting material of formula (II) in a vehicle, adding 0.001 to 3 equivalent of an oxidant, selectively adding a catalyst, and stirring the reaction for 30 minutes to 24 hours at 20° C. to 60° C. to obtain the compound of formula (I). Oxidants include, but are not limited to, one or more of: m-chloroperbenzoic acid, peracetic acid, potassium peroxymonosuifate (Oxone), cumene hydroperoxide, tert-butyl hydroperoxide, acetone butylperoxy, sodium periodate and hydrogen peroxide (H₂O₂); vehicles include, but are not limited to, one or more of: methanol, ethanol, methylene chloride, chloroform, tetrahydrofuran, acetonitrile, 1,2-dichloroethane, toluene and 1,4-dioxane; catalysts are selected from one or more groups of titanium tetraisopropanolate and L-(+)-diethyl tartrate, titanium tetraisopropanolate and D-(−)-diethyl tartrate, titanium tetraisopropanolate and (R,R)-1,2-diphenyl-1, 2-ethylene glycol or titanium tetraisopropanolate and (S,S)-1,2-diphenyl-1,2-ethylene glycol.

In a preferred embodiment, when L is a sulfoxide-containing structure fragment, the compound of formula (I) has a stereoisomer. The corresponding stereoisomer may be isolated by, for example, but not limited to, HPLC. For example, isolation with a silica gel column can be performed to obtain, but not limited to, compounds A1, A2, A3, B1, B2, B3, C1, C2, C3, C4, D1, D2, D3, E1, E2, F1, F2, F3, G, H and I.

The present invention also provides a pharmaceutical composition comprising an effective dose of the compound of formula (I) or its pharmaceutically acceptable salts, stereoisomers, or optical isomers, wherein said pharmaceutical composition is suitable for oral, rectal, parenteral, intranasal or transdermal administration or by inhalation or by suppository administration; said pharmaceutical composition is in the form of tablets, capsules, troches, lozenges, water or oil suspensions, dispersible powder, granules, or sublingual tablets.

The present invention further relates to the use of the compound of formula (I) or its pharmaceutically acceptable salts, stereoisomers, or optical isomers in the treatment of disorders resulting from cell proliferation and/or angiogenesis or disorders associated with or accompanying cell proliferation and/or angiogenesis, wherein the disorders are selected from: anti-proliferative disorders (e.g., cancer); neurodegenerative diseases, including: Huntington disease, polyglutamine disease, Parkinson's disease, Alzheimer's disease, epileptic seizures, striatum degeneration, progressive supranuclear palsy, insufficiency of torsional strain, spastic torticollis and dyskinesia, familial tremor, Tourette's syndrome, diffuse Lewy body disease (DLBD), Pick's disease, intracranial hemorrhage, primary lateral sclerosis, spinal muscular atrophy, amyotrophic lateral sclerosis, hypertrophic interstitial polyneuropathy, retinitis pigmentosa, hereditary optic atrophy, hereditary spastic paraplegia, progressive ataxia and Shy-Drager syndrome; metabolic diseases, including: type 2 diabetes; eye degenerative diseases, including: glaucoma, age-related macular degeneration, rubeotic glaucoma; inflammatory and/or immune system disorders, including: rheumatoid arthritis (RA), osteoarthritis, juvenile chronic arthritis, graft versus host disease, psoriasis, asthma, spondyloarthropathies, psoriasis, Crohn's disease, inflammatory bowel disease, colonic ulcer, alcoholic hepatitis, diabetes, Sjoegren's syndrome, multiple sclerosis, ankylosing spondylitis, membranous glomerulopathy, discogenic pain, systemic lupus erythematosus; diseases involving angiogenesis, including: cancer, psoriasis, rheumatoid arthritis; psychological disorders, including: bipolar disorder, schizophrenia, mania, depression and dementia; cardiovascular diseases, including: cardiac failure, restenosis and atherosclerosis; fibrotic diseases, including: hepatic fibrosis, cystic fibrosis and vascular fibrillation; infectious diseases, including: fungal infections, such as: candida albicans, bacterial infections, viral infections, such as: herpes simplex, protozoal infections, such as: malaria, leishmania infection, Trypanosoma brucei infection, toxoplasmosis and coccidiosis; and hematopoietic disorders, including: thalassemia, anemia and sickle-cell anemia.

The term “optionally substituted” used herein means that the group may be further substituted or fused by one or more non-hydrogen substituents. These substituents are independently selected from one or more of the following groups: halogen, ═O, ═S, —CN, —NO₂, —CF₃, —OCF₃, alkyl, haloalkyl, heteroalkyl, cycloalkyl, cycloalkenyl, heterocycloalkyl, aryl, heteroaryl, cycloalkylalkyl, arylalkyl, heteroarylalkyl, cycloalkylheteroalkyl, heterocycloalkylheteroalkyl, heteroarylheteroalkyl, arylheteroalkyl, hydroxy, alkoxy, hydroxyalkyl, amino, alkylamino, aminoalkyl, acylamino, alkylsulfonyl and acyl.

“Halogen” refers to fluorine, chlorine, bromine, and iodine.

As a group or part of a group, “alkyl” is a C1-C14 straight or branched chain aliphatic hydrocarbon group, unless otherwise indicated. Preferably, alkyl groups include C1-C6 alkyl, more preferably, methyl, ethyl, propyl, isopropyl, 3,3-dimethylbutyl, butyl, and pentyl, and particularly preferably isopropyl.

“Hetero atoms” means S, O, and N atoms.

“Heteroalkyl” refers to a straight-chain or branched chain containing alkyl group, containing one or more heteroatoms selected from S, O and N in the main chain. For heteroalkyl, a chain containing 2 atoms to 14 atoms is preferred. Heteroalkyl includes, but is not limited to, ethers, thioethers, alkyl esters, secondary or tertiary amines, and alkyl sulfinic acids.

“Cycloalkyl” refers to a saturated or partially saturated monocyclic, fused ring, or spiro-carbocyclic group. A cycloalkyl group consisting of 3 to 9 carbon atoms is preferred. The group may be a terminal group or a bridging group.

“Cycloalkenyl” refers to a non-aromatic monocyclic or polycyclic ring group. It contains at least one carbon-carbon double bond and each ring preferably has 5 to 10 carbon atoms. The group may be a terminal group or a bridging group.

“Heterocycloalkyl” refers to a cycloalkyl group containing at least one heteroatom, preferably containing 1 to 3 heteroatoms. A ring is preferably a 3-14 membered ring, more preferably a 4-7 membered ring. Cycloalkyl and heteroatom are as defined above. The group may be a terminal group or a bridging group.

“Aryl” refers to an optionally substituted monocyclic or fused ring, aromatic carbocyclic ring (aryl group may be substituted with one or more substituents) group, each ring preferably containing 5 to 12 carbon atoms (ring atoms are of the ring structure of carbon). The group may be a terminal group or a bridging group.

“Heteroaryl” refers to a group containing an aromatic ring, having one or more heteroatoms in the ring atom of the aromatic ring. Heteroatom is as defined above. The group may be a terminal group or a bridging group. The heteroaryl group may be substituted with one or more substituents.

“Cycloalkylalkyl” refers to cycloalkyl-alkyl, wherein the cycloalkyl and alkyl moieties are as described above. The group may be a terminal group or a bridging group.

“Arylalkyl” refers to: (aryl-alkyl) group, wherein aryl and alkyl are as defined herein. The group may be a terminal group or a bridging group.

“Heteroarylalkyl” refers to (heteroaryl-alkyl) group, wherein aryl and alkyl moieties are as defined herein. The group may be a terminal group or a bridging group.

“Arylheteroalkyl” refers to a (aryl-heteroalkyl) group, wherein aryl and heteroalkyl are as defined herein. The group may be a terminal group or a bridging group.

“Cycloalkylheteroalkyl” refers to a (cycloalkyl-heteroalkyl) group, wherein cycloalkyl and heteroalkyl are as defined herein. The group may be a terminal group or a bridging group.

“Heterocycloalkylheteroalkyl” refers to a (heterocycloalkyl-heteroalkyl) group, wherein heterocycloalkyl and heteroalkyl are as defined herein. The group may be a terminal group or a bridging group.

“Heteroarylheteroalkyl” refers to a (heteroaryl-heteroalkyl) group, wherein heteroaryl and heteroalkyl are as defined herein. The group may be a terminal group or a bridging group.

“Aminoalkyl” refers to a (amino-alkyl) group, wherein alkyl is as defined herein. The group may be a terminal group or a bridging group.

“Alkoxy” refers to —O-alkyl, wherein alkyl is as defined herein. The alkoxy group is preferably a C1-C6 alkoxy group. The group may be a terminal group or a bridging group.

“Cycloalkoxy” refers to —O-cycloalkyl, wherein cycloalkyl is as defined herein. The group may be a terminal group or a bridging group.

“Alkenyloxy” refers to —O-lower alkene. The group may be a terminal group or a bridging group.

“Alkynyloxy group” refers to —O-lower alkyne, wherein lower alkyne refers to C2-C6 alkyne. The group may be a terminal group or a bridging group.

“Aryloxy” refers to —O-aryl, wherein aryl is as defined herein. The group may be a terminal group or a bridging group.

“Heterocyclicalkoxy” refers to —O-heterocycloalkyl, wherein heterocycloalkyl is as defined herein. The group may be a terminal group or a bridging group.

Unless otherwise indicated, “alkylamino” refers to monoalkylamino and dialkylamino. “Monoalkylamino” refers to —NH-alkyl, where alkyl is as defined above. “Dialkylamino” refers to —N (alkyl)₂, wherein each alkyl may be the same or different, and are in line with the definition of alkyl herein. The group may be a terminal group or a bridging group.

Unless otherwise specified, “arylamino” includes monoarylamino and diarylamino. “Monoarylamino” represents formula aryl-NH—, wherein aryl is as defined above. Diarylamino represents formula (aryl)₂N—, wherein each aryl may be the same or different, and are in line with the definition of aryl herein. The group may be a terminal group or a bridging group.

“Acyl” represents alkyl-CO—, wherein alkyl is as defined herein. The group may be a terminal group or a bridging group.

“Sulfonyl” represents —S(O)₂—. The group may be a terminal group or a bridging group.

“Acylamino” represents a (acyl-amino) group, wherein acyl is as defined herein. The group may be a terminal group or a bridging group.

“Aminosulfonyl” represents a (amino-sulfonyl) group, wherein sulfonyl is as defined herein. The group may be a terminal group or a bridging group.

“Alkyl sulfonyl” refers to —S(O)₂-alkyl; “alkylsulfinyl” refers to a —SO-alkyl, wherein alkyl is as defined herein. The group may be a terminal group or a bridging group.

“Hydroxyalkyl” refers to a -alkyl-hydroxy group, wherein alkyl is as defined herein.

In one aspect, the present invention relates to a compound represented by formula (I) and its various possible isomers, including: diastereomers, enantiomers, stereoisomers, tautomers, and geometric isomers of “E” or “Z” configurational isomers. Any chemist with a certain basis can isolate the above optically pure or stereoisomeric compounds.

In another aspect, the present invention relates to a mixture of a compound represented by formula (I) and possible raceme or/and enantiomer or/and diastereomer thereof.

In one aspect, a compound represented by formula (I) in application, also covers the solvated and unsolvated forms of the compound. Thus, the present invention relates not only to compounds having the indicated configuration, but also to their hydrated and anhydrous forms.

In another aspect, in addition to a compound represented by formula (I) the present invention also relates to a pharmaceutically acceptable salt and prodrug thereof and an active metabolite of the compound and a pharmaceutically acceptable salt of the metabolite.

The term “pharmaceutically acceptable salts” refers to certain salts in which the above compounds are capable of maintaining the original biological activity and are suitable for pharmaceutical use. Pharmaceutically acceptable salts of a compound represented by formula (I) are formed in one of two forms: one is a salt formed with an acid; the other is a salt formed with an alkali or alkali metal. Acids for forming pharmaceutically acceptable salts with a compound represented by formula (I) include inorganic acids and organic acids. Suitable inorganic acids include: hydrochloric acid, sulfuric acid, and phosphoric acid. Suitable organic acids may be selected from aliphatic, cycloaliphatic, aromatic, heterocyclic carboxylic acids and sulfonic acid organic acids, examples of which include, but are not limited to, formic acid, acetic acid, propionic acid, succinic acid, glycolic acid, gluconic acid, lactic acid, malic acid, tartaric acid, glycine, arginine, citric acid, fumaric acid, alkylsulfonic acid, and arylsulfonic acid. Alkali metals for forming pharmaceutically acceptable salts with a compound represented by formula (I) include: lithium, sodium, potassium, magnesium, calcium, aluminum and zinc; alkalis for forming pharmaceutically acceptable salts with a compound represented by formula (I) include: choline, diethanolamine, and morpholine, etc.

“Prodrug” is a derivative of a compound represented by formula (I). It is converted in vivo by means of in vivo metabolism (e.g., by hydrolysis, reduction, or oxidation) into a compound represented by formula (I). For example, a compound represented by formula (I) containing a hydroxyl group can be reacted with an acid to prepare the corresponding ester. The corresponding ester is a prodrug that is converted by hydrolysis into the parent drug in vivo. Suitable acids for preparing “prodrugs” include, but are not limited to, acetic acid, citric acid, lactic acid, tartaric acid, malonic acid, oxalic acid, salicylic acid, succinic acid, fumaric acid, maleic acid, methylene-bis-beta-hydroxynaphthoic acid, gentisic acid, isethanesulfonic acid, methanesulfonic acid, ethanesulfonic acid, benzenesulfonic acid, and p-toluenesulfonic acid, etc.

### SPECIFIC EMBODIMENTS

In the following embodiments, unless otherwise specified, all the temperatures are in degrees Celsius.

Romidepsin of the present invention is prepared using the method of Patent CN103173390A by microbial fermentation (Bai Hua et al., A Chromobacteria Strain and Application Thereof: CN103173390A [P]. 2013-06-26), trisulfide cyclic peptides of the present invention is prepared by the method described in Patent CN104072589A (Bai Hua et al., Antineoplastic Sulfur Compound and Its Preparation and Application: CN104072589A [P]. 2014-10-01). Various other starting materials and reagents are commercially available. Suppliers include, but are not limited to: Aldrich Chemical Company, Lancaster Synthesis Ltd, and so on. Unless otherwise specified, commercially available materials and reagents are used without further purification. Glassware is dried using an oven and/or by heating.

¹H-NMR spectrum is obtained by measurement with a Bruker instrument (400 MHz), with chemical shifts expressed in ppm. Chloroform is used as the reference standard (7.25 ppm) or tetramethylsilane internal standard (0.00 ppm). If desired, other common NMR vehicles can also be used. Representation in ¹H NMR: s=singlet, d=doublet, t=triplet, m=multiplet, br=broadened, dd=doublet of doublet, dt=doublet of triplet. If a coupling constant is provided, its unit is Hz.

High-resolution mass spectra are obtained by measurement using a Bruker instrument (micrOTOF-QII); the ionization mode may be ESI.

The following examples are merely illustrative of the synthetic methods of the specific compounds to be protected, and there is no restriction on the synthesis method. Compounds not listed in the embodiments can also be prepared by adjusting the usual reaction conditions with appropriate synthetic conditions such as synthetic routes and synthetic methods as described below, by selecting appropriate starting materials, where adjustments are necessary.

### Embodiment 1

Synthesis of Compounds A and C

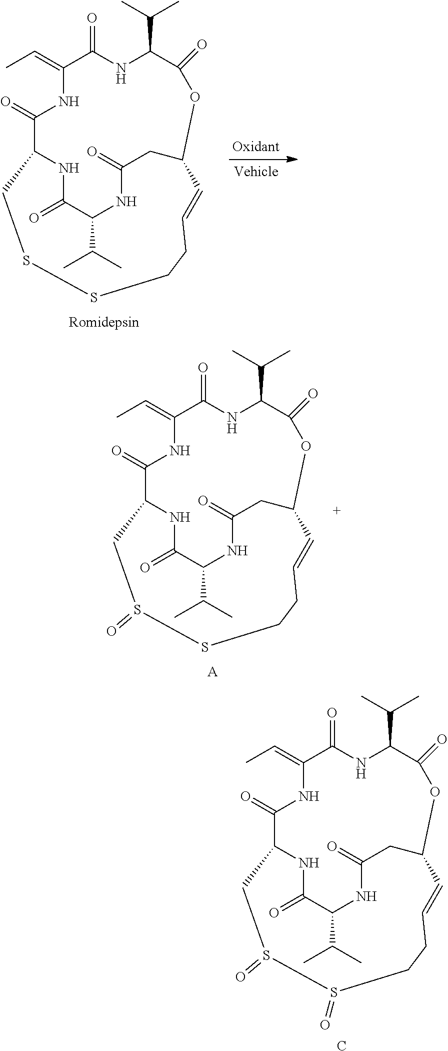

1 g of Romidepsin was dissolved in 10 ml of dichloromethane; 0.38 g of 3-Chloroperbenzoic acid (MCPBA) was added; at 0° C., the reaction was stirred for 2 hours; the reaction end point was monitored with HPLC. After completion of the reaction, HPLC (isolation conditions: mobile phase: A: water B: acetonitrile (2 min: A 80%, B 20%; 16 min: A20%, B 80%; 18 min: A 20%, B 80%; 18.2 min: A 80%, B 20%; 21 min: A 20%, B 80%); column: C18, 5 um, 21.2×150 mm; flow rate: 10 ml/min; detection wavelength: w=214) was used for separation to respectively obtain compound A and compound C.

¹H-NMR spectrum data of compound A are as follows:

¹H-NMR (400 MHz, (CD₃)₂CO): δ 8.67 (1H, s), 7.67 (1H, d, J=3.7), 7.56 (1H, d, J=9.8), 7.04 (1H, d, J=8.4), 6.75 (1H, q, J=7.2), 5.80-5.81 (2H, m), 5.64 (1H, d, J=6.7), 5.44-5.48 (1, m), 4.65 (1H, dd, J₁=4.7, J₂=8.6), 4.11 (1H, t, J=9.4), 3.71 (1H, dd, J=4.5, J=14.2), 3.42-3.50 (3H, m), 2.98 (1H, d, J=13.5), 2.72-2.81 (2H, m), 2.52-2.58 (1H, m), 2.29-2.37 (2H, m), 1.75 (3H, d, J=7.1), 1.07 (3H, d, J=7.3), 0.98 (3H, d, J=7.0), 0.96 (3H, d, J=7.0).

¹H-NMR spectrum data of compound C are as follows:

¹H-NMR (400 MHz, DMSO): δ 8.63 (1H, s), 8.0 (1H, d, J=6.2), 7.30 (1H, d, J=7.0), 6.99 (1H, d, J=9.2), 6.39 (1H, q, J=6.9), 5.67-5.80 (2H, m), 5.56 (1H, d, J=5.6), 5.24-5.27 (1, m), 4.27 (1H, dd, J=4.3, J=11.3), 4.15 (1H, t), 4.03 (1H, s), 3.33-3.41 (3H, m), 3.29 (1H, d, J=13.0), 2.61-2.67 (1H, m), 2.40-2.43 (2H, m), 2.28-2.32 (1H, m), 1.57 (1H, d, J=6.7), 1.01 (3H, d, J=6.9), 0.97 (3H, d, J=6.9), 0.93 (3H, d, J=6.8), 0.87 (3H, d, J=6.8).

Compound A and compound C were isolated by HPLC (isolation conditions: mobile phase A: water/acetonitrile/trifluoroacetic acid=90/10/0.1, B: acetonitrile/water=90/10 (5 min: A 15%, B 85%; 55 min: A 25%, B 75%; 55.5 min: A 25%, B 75%; 56 min: A 15%, B85%; 61 min: A 15%, B 85%); column: C18, 5 um, 21.2×150 mm; flow rate: 10 ml/min; detection wavelength: w=214) to obtain isomers of compounds A and C (A1, A2, A3, C1, C2, C3, C4). Other structural identification data of compounds A and C and isomers thereof are shown in Table 1.

### Embodiment 2

Synthesis of Compound B

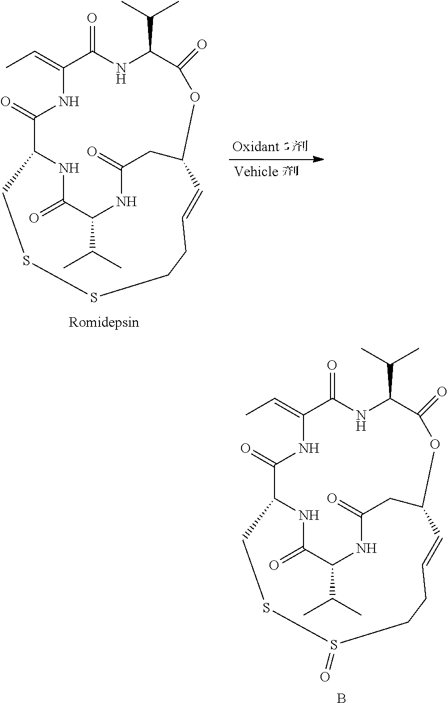

In a 500 mL round bottom flask, 200 mL of dichloromethane (DCM) was added, followed by addition of 2.27 g (2.37 mL) of titanium tetraisopropanolate and 3.30 g (2.74 mL) of L-(+)-diethyl tartrate, thoroughly stirred in an ice bath; 0.14 mL of water was added; 4.32 g of Romidepsin was added after stirring for 10 minutes, and the mixture was thoroughly stirred in an ice bath; 4.23 mL of cumene hydroperoxide with a content greater than 70% was added, the ice bath was removed after the addition was completed; the mixture was stirred at room temperature. After 5 hours, HPLC was used to detect the reaction end point. After the reaction, 15 mL of water was added, and the DCM in the reaction solution was removed under reduced pressure to obtain an amber viscous precipitate. After 15 mL of acetone was added and the mixture stirred evenly, the mixture was poured into centrifuge tubes for centrifugation (6000 r/min). After stratification, the supernatant was collected. After acetone was added to the residue for washing, the centrifugation was performed again, and the supernatant was collected after stratification. The supernatants mentioned above were combined as a preparation sample, and HPLC was used for preparative isolation. Preparation conditions: (mobile phase: A: water B: acetonitrile (2 min: A 80%, B 20%; 16 min: A 20%, B 80%; 18 min: A 20%, B 80%; 18.2 min: A 80%, B 20%; 21 min: A 20%, B 80%); column: C18, 5 um, 21.2×150 mm; flow rate: 10 ml/min; detection wavelength: w=214). After the preparation, compound B was obtained.

¹H-NMR spectrum data of compound B are as follows:

¹H-NMR (400 MHz, DMSO): δ 9.05 (1H, s), 7.70 (1H, d, J=6.4), 7.15 (1H, d, J=8.4), 6.76 (1H, d, J=9.6), 6.50 (1H, q, J=6.8), 6.02-6.08 (1H, m), 5.53-5.56 (2H, m), 5.22-5.25 (1, m), 4.36 (1H, dd, J=5.5, J=8.2), 4.27 (1H, dd, J=3.9, J=6.1), 3.50-3.59 (2H, m), 3.23-3.28 (1H, m), 3.09 (1H, dd, J=3.1, J=15.5), 3.03 (1H, d, J=13.3), 2.67-2.70 (1H, m), 2.58 (1H, dd, J₁=6.9, J₂=13.3), 2.45-2.55 (1H, m), 2.37-2.42 (1H, m), 2.15-2.20 (1H, m), 1.59 (3H, d, J=7.1), 0.96 (6H, d, J=6.9), 0.93 (3H, d, J=6.8), 0.89 (3H, d, J=6.8).

Compound B was isolated by HPLC (isolation conditions: mobile phase:

A: Water/acetonitrile/trifluoroacetic acid=90/10/0.1, B: Acetonitrile/water=90/10 (5 min: A 15%, B 85%; 55 min: A25%, B 75%; 55.5 min: A25%, B 75%; 56 min: A15%, B85%; 61 min: A 15%, B 85%); column: C18, 5 um, 21.2×150 mm; flow rate: 10 ml/min; detection wavelength: w=214) to obtain isomers (B1, B2, B3) of compound B. Other structural identification data of compound B and isomers thereof are shown in Table 1.

### Embodiment 3

Synthesis of Compounds D, E, and F

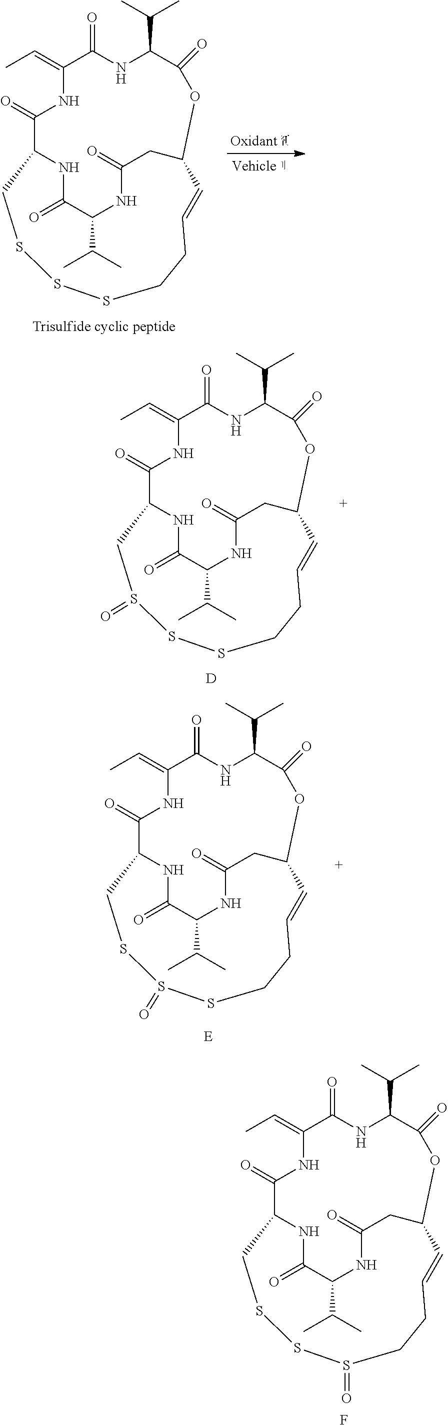

1 g of trisulphide cyclic peptide was dissolved in 10 ml of dichloromethane; 0.38 g of 3-Chloroperbenzoic acid (MCPBA) was added; at 0° C., the reaction was stirred for 2 hours; the reaction end point was monitored with HPLC. After completion of the reaction, the HPLC was used (isolation conditions: mobile phase: A: water, B: acetonitrile (2 min: A 80%, B 20%; 16 min: A 20%, B 80%; 18 min: A 20%, B 80%; 18.2 min: A 80%, B 20%; 21 min: A20%, B 80%); column: C18, 5 um, 21.2×150 mm; flow rate: 10 ml/min; detection wavelength: w=214) for isolation to respectively obtain compound D, compound E, and compound F. Compound D, compound E, and compound F were isolated by HPLC (isolation conditions: mobile phase: A: water/acetonitrile/trifluoroacetic acid=90/10/0.1, B: acetonitrile/water=90/10 (5 min: A 15%, B 85%; 55 min: A 25%, B 75%; 55.5 min: A 25%, B 75%; 56 min: A15%, B85%; 61 min: A15%, B 85%); column: C18, 5 um, 21.2×150 mm; flow rate: 10 ml/min; detection wavelength: w=214) to obtain isomers (D1, D2, D3, E1, E2, F1, F2, F3) of compounds D, E, and E Structural identification data of compounds D, E, and F and isomers thereof are shown in Table 1.

### Embodiment 4

Synthesis of Compounds G, H, and I

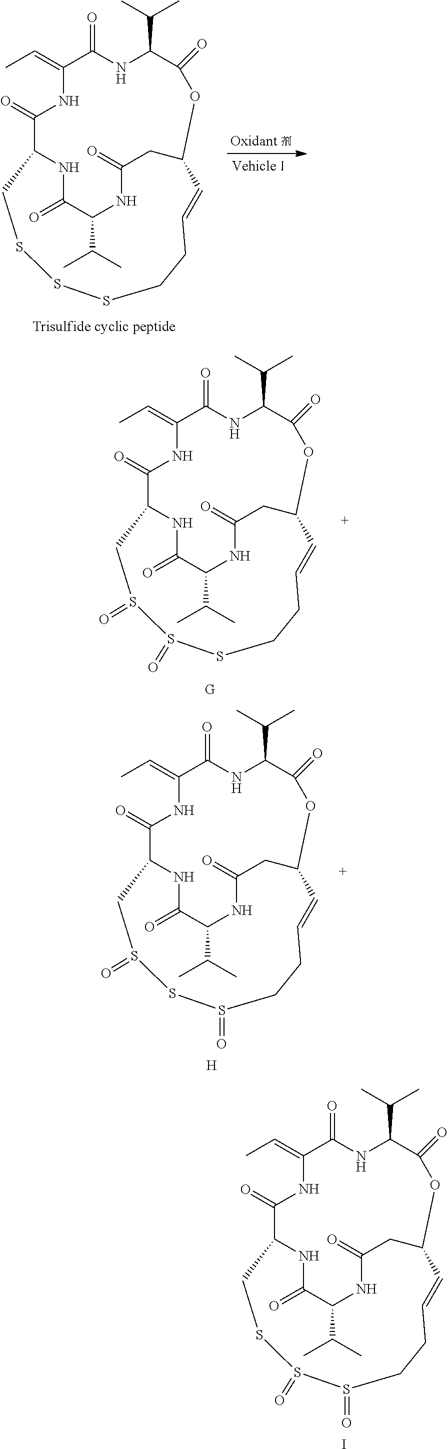

1 g of trisulphide cyclic peptide was dissolved in 10 ml of dichloromethane; 0.95 g of 3-Chloroperbenzoic acid (MCPBA) was added; at 0° C., the reaction was stirred for 2 hours; the reaction end point was monitored with HPLC. After completion of the reaction, HPLC was used (isolation conditions: mobile phase: A: water, B: acetonitrile (2 min: A 80%, B 20%; 16 min: A 20%, B 80%; 18 min: A 20%, B 80%; 18.2 min: A 80%, B 20%; 21 min: A20%, B 80%); column: C18, 5 um, 21.2×150 mm; flow rate: 10 ml/min; detection wavelength: w=214) for isolation to respectively obtain compound G, compound H, and compound I. Structural identification data of compounds G, H, and I are shown in Table 1.

TABLE 1
Structures and structural identification data of compounds
of the present invention

| Compound | Structural formula | [M + H] |
|---|---|---|
| A | 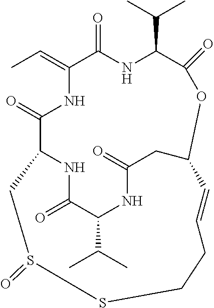 [SMILES: C/C=C1\NC(=O)[C@H]2CS(=O)SCC/C=C/[C@H](CC(=O)N[C@H](C(C)C)C(=O)N2)OC(=O)[C@H](C(C)C)NC1=O] | 557.7 |
| A1 | 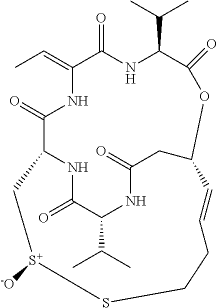 | 557.7 |
| A2 | 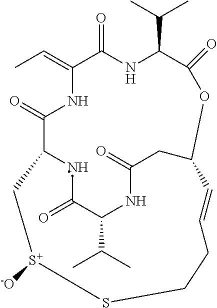 | 557.7 |
| A3 | 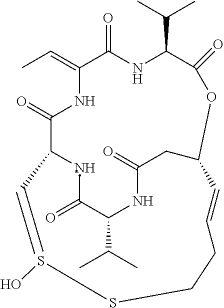 [SMILES: C/C=C1\NC(=O)[C@H]2/C=S(/O)SCC/C=C/[C@H](CC(=O)N[C@H](C(C)C)C(=O)N2)OC(=O)[C@H](C(C)C)NC1=O] | 557.7 |
| B | 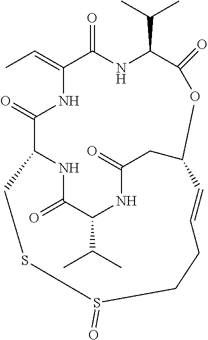 [SMILES: C/C=C1\NC(=O)[C@H]2CSS(=O)CC/C=C/[C@H](CC(=O)N[C@H](C(C)C)C(=O)N2)OC(=O)[C@H](C(C)C)NC1=O] | 557.7 |
| B1 | 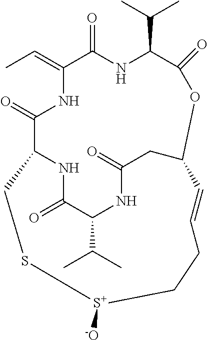 | 557.7 |
| B2 | 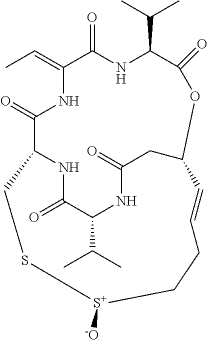 | 557.7 |
| B3 | 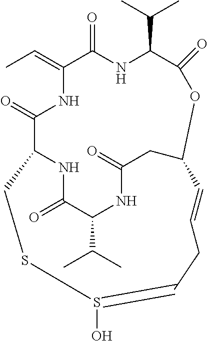 [SMILES: C/C=C1\NC(=O)[C@H]2CS/S(O)=C\C/C=C/[C@H](CC(=O)N[C@H](C(C)C)C(=O)N2)OC(=O)[C@H](C(C)C)NC1=O] | 557.7 |
| C | 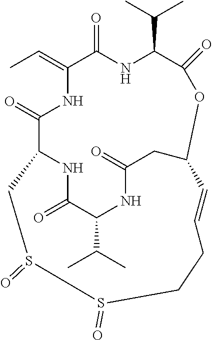 [SMILES: C/C=C1\NC(=O)[C@H]2CS(=O)S(=O)CC/C=C/[C@H](CC(=O)N[C@H](C(C)C)C(=O)N2)OC(=O)[C@H](C(C)C)NC1=O] | 573.7 |
| C1 | 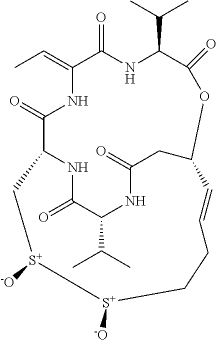 | 573.7 |
| C2 | 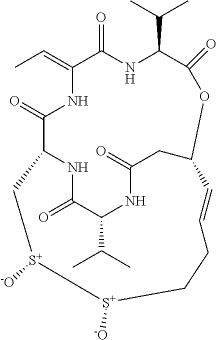 | 573.7 |
| C3 | 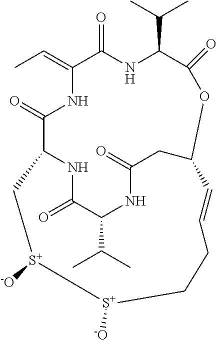 | 573.7 |
| C4 | 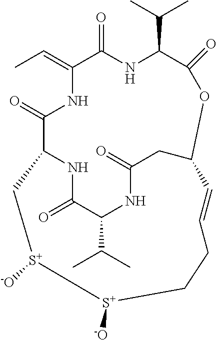 | 573.7 |
| D | 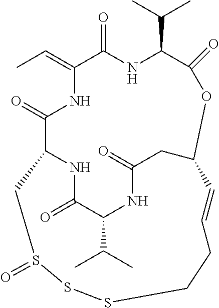 [SMILES: C/C=C1\NC(=O)[C@H]2CS(=O)SSCC/C=C/[C@H](CC(=O)N[C@H](C(C)C)C(=O)N2)OC(=O)[C@H](C(C)C)NC1=O] | 589.8 |
| D1 | 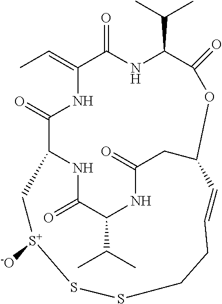 | 589.8 |
| D2 | 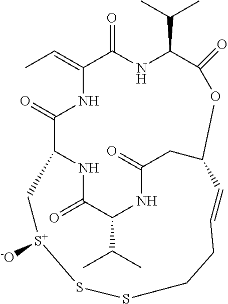 | 589.8 |
| D3 | 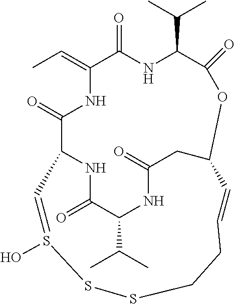 [SMILES: C/C=C1\NC(=O)[C@H]2/C=S(/O)SSCC/C=C/[C@H](CC(=O)N[C@H](C(C)C)C(=O)N2)OC(=O)[C@H](C(C)C)NC1=O] | 589.8 |
| E | 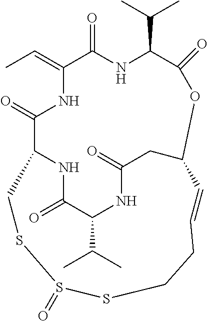 [SMILES: C/C=C1\NC(=O)[C@H]2CSS(=O)SCC/C=C/[C@H](CC(=O)N[C@H](C(C)C)C(=O)N2)OC(=O)[C@H](C(C)C)NC1=O] | 589.8 |
| E1 | 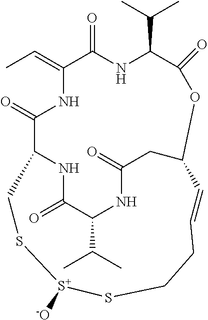 | 589.8 |
| E2 | 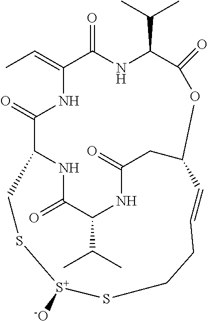 | 589.8 |
| F | 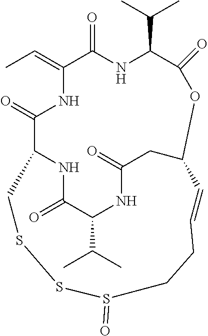 [SMILES: C/C=C1\NC(=O)[C@H]2CSSS(=O)CC/C=C/[C@H](CC(=O)N[C@H](C(C)C)C(=O)N2)OC(=O)[C@H](C(C)C)NC1=O] | 589.8 |
| F1 | 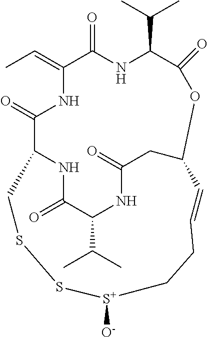 [SMILES: C/C=C1\NC(=O)[C@H]2CSS[S@+]([O-])CC/C=C/[C@H](CC(=O)N[C@H](C(C)C)C(=O)N2)OC(=O)[C@H](C(C)C)NC1=O] | 589.8 |
| F2 | 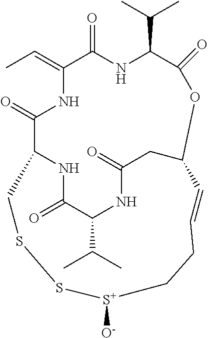 [SMILES: C/C=C1\NC(=O)[C@H]2CSS[S@+]([O-])CC/C=C/[C@H](CC(=O)N[C@H](C(C)C)C(=O)N2)OC(=O)[C@H](C(C)C)NC1=O] | 589.8 |
| F3 | 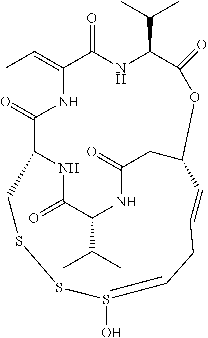 [SMILES: C/C=C1\NC(=O)[C@H]2CSS/S(O)=C\C/C=C/[C@H](CC(=O)N[C@H](C(C)C)C(=O)N2)OC(=O)[C@H](C(C)C)NC1=O] | 589.8 |
| G | 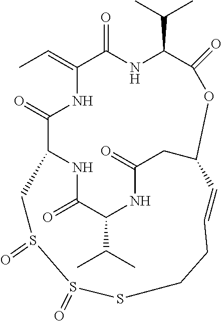 [SMILES: C/C=C1\NC(=O)[C@H]2CS(=O)S(=O)SCC/C=C/[C@H](CC(=O)N[C@H](C(C)C)C(=O)N2)OC(=O)[C@H](C(C)C)NC1=O] | 604.7 |
| H | 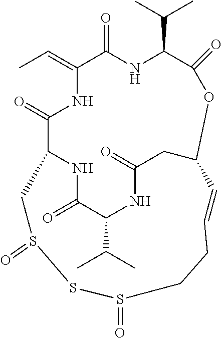 [SMILES: C/C=C1\NC(=O)[C@H]2CS(=O)SS(=O)CC/C=C/[C@H](CC(=O)N[C@H](C(C)C)C(=O)N2)OC(=O)[C@H](C(C)C)NC1=O] | 604.7 |
| I | 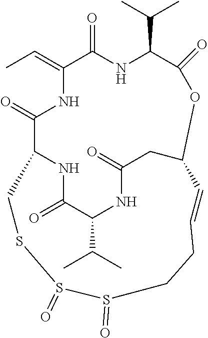 [SMILES: C/C=C1\NC(=O)[C@H]2CSS(=O)S(=O)CC/C=C/[C@H](CC(=O)N[C@H](C(C)C)C(=O)N2)OC(=O)[C@H](C(C)C)NC1=O] | 604.7 |

The in vitro and in vivo anti-tumor test studies on the compounds of the present invention are provided below.

I. Tumor Cell Growth Inhibition Activity Study

The biological efficacy of the compounds of the present invention is demonstrated by in vitro cell activity. The tetrazolium salt (MTT) reduction method was used to test the activity of the compounds of the present invention. Human breast cancer cells (MDA-MB-231) (CAS cell library; catalog number: TCHu227), human lung cancer cells (A549) (ATCC® Number: CRM-CCL-185™), human colon cancer cells (HCT-116) (CAS cell library; catalog number: TCHu99), acute monocytic leukemia cells (MV4-11) (ATCC® Number: CRL-9591™), tissue cell lymphoma cells (U-937) (CAS cell library; catalog number: TCHu159), human acute promyelocytic leukemia cells (HL-60) (ATCC® Number: CCL-240™), chronic myelogenous leukemia cells (of K562) (CAS cell library; catalog number: TCHu191), human hepatocellular carcinoma cells (BEL-7404) (CAS cell library; catalog number: TCHu64), human pancreatic cancer cells (BxPC3) (ATCC® Number: CRL-1687™)) and human prostate cancer cells (PC-3) (ATCC®Number: CRL-1687™) were inoculated in 96-well plates, 90 μL/well, with 3000 to 8000 cells in respective well; in the first row of plate, 100 μL/well culture medium was added; the 96-well plate was pre-incubated in an incubator at 37° C., 5% CO₂, and 100% relative humidity for 24 hours.

A compound was weighed and configured to a 10 mM/L stock solution, and the compound was diluted three-fold sequentially from the highest concentration of 2 millimoles (mM); 95 μl the corresponding cell medium was added to each well of a V-bottom dispensing plate; 5 μl of the compound on a 200× plate was transferred to a 10× dispensing plate according to the concentration; 5 μl of dimethylsulfoxide (DMSO) was evenly mixed in the corresponding 95 μl cell culture medium as a vehicle control. 10 μl of the compound-medium in each well of the 10× dispensing plate was added to a 96-well plate already plated with cells. The 10 μl of vehicle control was added to blank control wells and vehicle control wells. The cell culture plates were returned to the incubator for 72 hours.

After the compound for cells was treated for 72 hours, 20 microliters of 5 mg/ml MTT was added to each well; all the steps need to be aseptic. The 96-well plate was incubated in a 37° C. and 5% CO2 incubator for 4 hours. The media was carefully aspirated, and 100 μl of DMSO was added; the 96-well plate was properly packaged with a foil. The plate was shaken with a shaker for 20 minutes to induce cell lysis. The absorbance at 490 nm was read on a SpectraMax M2e instrument. The compound activity (relative IC50) was calculated using GraphPad Prism software.

TABLE 2
In vitro activity data for compounds of the present invention

| | IC50 (nM) | | |
|---|---|---|---|
| Cell line | Compound A | Compound B | Romidepsin |
| MDA-MB-231 | 9.7 | 2.0 | 3.6 |
| A549 | 10.7 | 3.6 | 5.2 |
| HCT-116 | 29.3 | 6.1 | 6.5 |
| MV4-11 | 33 | 4.8 | 6.8 |
| U-937 | 22.8 | 5.4 | 7.2 |
| HL-60 | 35.9 | 3.4 | 8.6 |
| K562 | 23.2 | 2.6 | 4.1 |
| BEL-7404 | 10 | 4.9 | 6.4 |
| BxPC3 | 13.9 | 6.5 | 8.7 |
| PC-3 | 11.9 | 6.1 | 7.1 |

In vitro activity data showed that: The present invention utilizes Romidepsin as a positive control drug; Romidepsin can inhibit tumor cell proliferation, with IC50 between 3.6 nM and 8.7 nM; compound A and compound B can inhibit proliferation of tumor cells, and the tumor inhibiting activity of compound B is better than that of Romidepsin; its IC50 is between 2.0 nM and 6.5 nM; the IC50 of compound A is between 9.7 nM and 35.9 nM.

II. In Vivo Pharmacodynamic Studies on Compound A

1. Establishment of an Animal Model

Female BALB/C nude mice 6 weeks to 8 weeks old and weighing about 19 g to 29 g were taken (Supplier: Beijing Vital River Laboratory Animal Technology Co., Ltd.) and raised for a week. A human cancer nude mice allogeneic transplanted tumor model was established: Human breast cancer cell line MDA-MB-231 (ATCC® Number: HTB-26™) and human lung cancer cell line NCI-H460 (ATCC® Number: HTB-177™) were incubated. The monolayer tumor cells were digested and detached; then, they were collected and resuspended in a serum-free culture medium, and then were adjusted to a concentration of 2×10⁶/0.2 mL. They were put into an iced box and brought to an animal house. A syringe with a No. 6 needle was used to directly take a 0.2 mL cell suspension and transplanted it into the rear subcutaneous scapular regions of the left axillaries of the nude mice at 2×10⁶/0.2 mL per mouse. Tumor volumes were measured once every 2 days to 3 days. After two weeks, tumor-bearing nude mice with vigorous tumor growth and no ulceration were selected, the tumors were removed from them under sterile conditions. The tumor tissues were cut to about 2 mm to 3 mm in diameter and inoculated in the rear subcutaneous scapular regions of the left axillaries of the nude mice. After passage for three generations, when the tumor volume grew to 100 mm³, nude mice with tumor masses that were too large or too small were removed and randomly grouped for administration.

2. Experimental Method

The mice were randomly divided into five groups, including the negative control group (vehicle), positive control group (Romidepsin, 2.4 mg/kg), treatment group with the high, medium, and low doses of compound A (respectively 7.2 mg/kg, 4.8 mg/kg, 2.4 mg/kg, wherein the high dose was lower than the maximum tolerated dose, MTD); there were six nude mice in each group; intravenous administration was given every 4 days; 4 doses were given. During the period, the weights and tumor volumes of the animals were measured every 2 days, and the number of animal deaths was recorded. 24 hours after the last administration, the animals were sacrificed. The tumor volumes, tumor weights, and body weights of nude mice were measured; tumor volume growth curves, nude mice weight growth curves and tumor inhibition rates, mortality of the animals were plotted; the relative tumor proliferation rate T/C (%) was calculated; according to the formula T/C (%)=TRTV/CRTV*100%. (TRTV: treatment group RTV; CRTV: negative control group RTV, relative tumor volume RTV=Vₜ/V₀, wherein V₀ is the tumor volume at the time of administration by group; Vₜ is the post-administration tumor volume).

3. Efficacy Results

By in vivo efficacy screening experiments, Romidepsin was used as a positive drug. The results showed that compound A had a good effect of inhibiting the growth of tumors in vivo; in addition, there was a dose-effect relationship; at the same dose, its antitumor effect was equivalent to that of Romidepsin. The screening results are shown in Table 3.

TABLE 3
Inhibition rates of compound A for the nude mice tumor model

| | Cell line | Compound A | Romidepsin |
|---|---|---|---|
| Relative | MDA-MB-231 | 45.5%(2.4 mg/kg, q4d, 4x) | 48.9%(2.4 mg/kg, q4d, 4x) |
| tumor | | 35.4%(4.8 mg/kg, q4d, 4x) | |
| proliferation | | 29.8%(7.2 mg/kg, q4d, 4x) | |
| rates T/C | NCI-H460 | 44%(2.4 mg/kg, q4d, 4x) | 42.2%(2.4 mg/kg, q4d, 4x) |
| (%) for | | 38.6%(4.8 mg/kg, q4d, 4x) | |
| different | | 25.2%(7.2 mg/kg, q4d, 4x) | |
| tumor | | | |
| models | | | |

Note:
Values in parentheses represent the doses, time intervals, and number of doses (mg/kg, dosing interval 4 days, 4 times). For different models, if T/C (%) <60%, it can be determined that this compound is effective for the model; the administration mode is intraperitoneal injection. The vehicle is 1% ethanol + 4% propylene glycol.

### III. Effect of Compound A on Blood Phase

1. Experimental Method

BAB-C mice (6 to 8 weeks old, a half being males and the other half females, weighing about 18 g-25 g, supplier: Beijing Vital River Laboratory Animal Technology Co., Ltd.) were raised for one week and then divided into three groups: normal control group, Romidepsin group, and compound A group. The normal group was injected with the vehicle; the drug group was injected with 0.5 mg/kg of the drug; 24 hours and 48 hours respectively after administration, a complete blood count (CBC) analysis was performed; for all the data, mean±standard deviation (mean±SD) was adopted; T tests were performed between inter-group data.

2. Experimental Results

The CBC analysis showed that after Romidepsin was administered, there was a significant effect of killing white blood cells (WBCs). At 24 h, the WBC count had decreased by 75%. At 48 h, it was restored to 40% of the normal group. At 24 h and 48 h with compound A, the WBCs showed no significant difference compared with the normal group. The experimental results indicated that compound A had no effect on WBCs in vivo; after Romidepsin and compound A were administered, the animals' PLT at 24 h and 48 h significantly decreased when compared with the normal group; there was no significant difference between the Romidepsin group and the compound A group (the results are shown in Table 4). The experiment results show that, when used as a chemotherapeutic agent for the treatment of a tumor, compound A, compared with Romidepsin, will not lead to the side effect of leucopenia or kill any immune cell of the body.

TABLE 4
Effect of compound A on blood cells

| | WBC (1 × 10*9/L) | | RBC (1 × 10*12/L) | | PLT (1 × 10*9/L) | |
|---|---|---|---|---|---|---|
| Group | 24 h | 48 h | 24 h | 48 h | 24 h | 48 h |
| Normal | 13.39 ± 3.46 | 14.64 ± 2.86 | 10.37 ± 0.61 | 10.23 ± 0.38 | 715.06 ± 111.70 | 695.63 ± 89.16 |
| group | | | | | | |
| Romidepsin | 4.32 ± 2.61** | 6.92 ± 3.29** | 10.30 ± 0.47 | 10.23 ± 0.78 | 600.56 ± 71.51** | 431.31 ± 64.96** |
| Compound A | 14.19 ± 2.55 | 14.50 ± 3.26 | 10.44 ± 0.46 | 10.42 ± 0.46 | 570.13 ± 71.36** | 483.00 ± 55.59** |

*P < 0.05 vs normal group;
**p < 0.01 vs normal group

IV. In Vivo Efficacy Study on Compound B

1. Establishment of an Animal Model

Female BALB/C nude mice 6 weeks to 8 weeks old and weighing about 19 g to 29 g were taken and raised for one week (supplier: Beijing Vital River Laboratory Animal Technology Co., Ltd.) MDA-MB-231 (ATCC® Number: HTB-26™) breast cancer cells were cultured in vitro; the culture conditions were: 10% fetal bovine serum was added to an L-15 medium, 37° C., and 0% CO₂. Passage was performed twice a week. When the cells were in the exponential growth phase, the cells were collected, counted, and inoculated. The right shoulders of each mouse were inoculated subcutaneously with 0.2 ml of MDA-MB-231 tumor cells (5×10²/ml). The cells were suspended in phosphate buffered saline (PBS) and Matrigel (1:1) was added. A total of 50 mice were inoculated. On day 4 after tumor cell inoculation, administration was started when the average tumor volume reached approximately 140 mm³.

2. Experimental Method

30 tumor-bearing mice were selected and divided into 5 groups by tumor volume, each group containing 6 mice. For the negative control group (vehicle), the positive control group (Romidepsin, 6.6 mg/kg), treatment group with the high, medium, and low doses of compound B (respectively 6.6 mg/kg, 4.4 mg/kg, and 2.2 mg/kg), intravenous injection was administered, once a week and for four weeks. The experimental indicator was to examine whether the tumor growth was inhibited, delayed, or subsided. Tumor diameter was measured three times a week with a vernier caliper. The formula for calculating tumor volume is as follows: V=0.5a×b², wherein a and b respectively indicate the major diameter and minor axis of the tumor. The antitumor efficacy of the compound was evaluated with T/C (%) and of TGI. T/C (%)=T_{RTV}/C_{RTV}*100% wherein T_{RTV} and C_{RTV} respectively indicate the relative tumor volumes of the treatment group and the vehicle control group; if T/C %≦40%, the drug is deemed to be effective; if T/C %≦10%, the drug is deemed to be very effective. TGI (%)=[1−(Ti−T0)/(Vi−V0)]×100; wherein Ti indicates the mean tumor volume of the treatment group on the specified date; T0 indicates the mean tumor volume of the treatment group at the time of administration by cage; Vi indicates the mean tumor volume of the vehicle group on the same day; V0 indicates the mean tumor volume of the vehicle group at the time of administration by cage. At the end of the experiment, the tumors were stripped, weighed, and photographed.

3. Efficacy Results

This experiment evaluated the efficacy of Romidepsin and compound B in the treatment of BALB/c nude mice model with xenografted MDA-MB-231 breast cancer. On day 28 after the administration, the tumor volume of the vehicle control group reached the 1189 mm³. Compared with the vehicle control group, the compound B 2.2 mg/kg, 4.4 mg/kg, and 6.6 mg/kg group exhibited a dose-dependent antitumor effect and a significant inhibitory effect after treatment. The mean tumor volumes of the three groups on day 28 were respectively 259 mm³ (T/C=11.3%, p<0.001), 202 mm³ (T/C=5.9%, p<0.001), 187 mm³ (T/C=4.5%, p<0.001); the compound B 6.6 mg/kg group, compared with the Romidepsin 6.6 mg/kg group (the mean tumor volume on day 28 day was 260 mm24]3 (T/C=11.4%, p<0.001), compound B had higher efficacy than Romidepsin did, and the efficacy of the compound B 2.2 mg/kg group was equivalent to that of the Romidepsin 6.6 mg/kg group.

TABLE 5
Tumor volumes, T/C values, and TGI values on day 28

| | Day 28 tumor | | | |
|---|---|---|---|---|
| | volume | T/C | TGI | Significant |
| Treatment group | (mm³)^{a} | (%) | (%) | difference |
| Vehicle | 1189 ± 160 | — | — | — |
| Romidepsin 6.6 mg/kg | 260 ± 103 | 11.4 | 88.6 | p < 0.001 |
| Compound B 2.2 mg/kg | 259 ± 47 | 11.3 | 88.7 | p < 0.001 |
| Compound B 4.4 mg/kg | 202 ± 38 | 5.9 | 94.1 | p < 0.001 |
| Compound B 6.6 mg/kg | 187 ± 29 | 4.5 | 95.5 | p < 0.001 |

^{a}Mean ± standard error

All the documents mentioned in the present invention are herein incorporated by reference as if each reference were individually incorporated by reference above. Further, it should be understood that, after reading the above teachings of the present invention, those skilled in the art may make various changes or modifications to the present invention, and these equivalents also fall within the scope as defined by the Claims attached to the present application.

1. A compound represented by formula (I) or a pharmaceutically acceptable salt, stereoisomer, or optical isomer thereof: 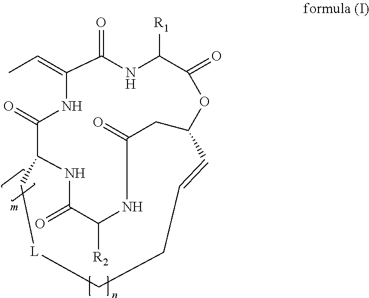
Wherein: n=0, 1 or 2; m=0 or 1;
R₁ and R₂ are independently selected from H, alkyl, nitro, cyano, halo, haloalkyl, haloalkenyl, hydroxy, hydroxyalkyl, alkoxy, alkoxycarbonyl, aryloxy, alkenyloxy, alkynyloxy, cycloalkoxy, heterocycloalkoxy, amino, alkylamino, aminoalkyl, amide group, alkylaminocarbonyl, sulfonyl, alkylsulfonyl, alkylsulfinyl, aminosulfonyl, acyl, cycloalkyl, cycloalkenyl, heterocycloalkyl, aryl, heteroaryl, cycloalkylalkyl, arylalkyl, heteroarylalkyl, cycloalkylheteroalkyl, heterocycloalkylheteroalkyl, heteroarylheteroalkyl, or arylheteroalkyl;
L is selected from:

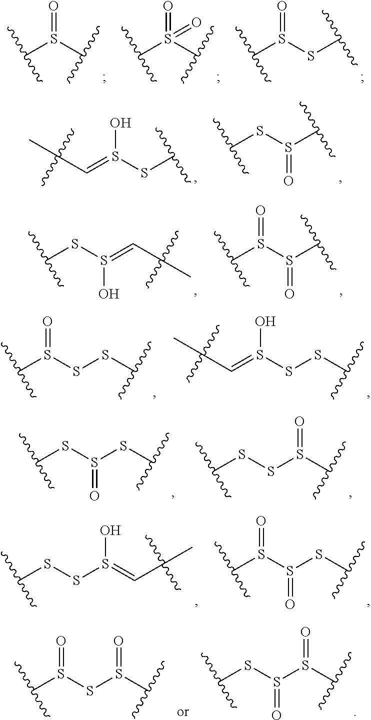

2. The compound as claimed in claim 1, wherein, R₁ and R₂ are independently selected from H, C₁-C₆ alkyl or C₁-C₆ alkoxy.

3. The compound as claimed in claim 1, wherein, L is selected from:

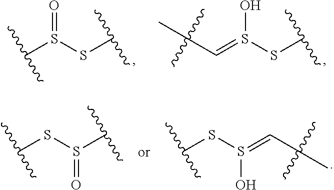 [SMILES: *SS(*)=O ; */C=S(\O)S* ; *SS(*)=O ; */C=S(\O)S*]

4. The compound as claimed in claim 1, wherein said compound represented by formula (I) is selected from:

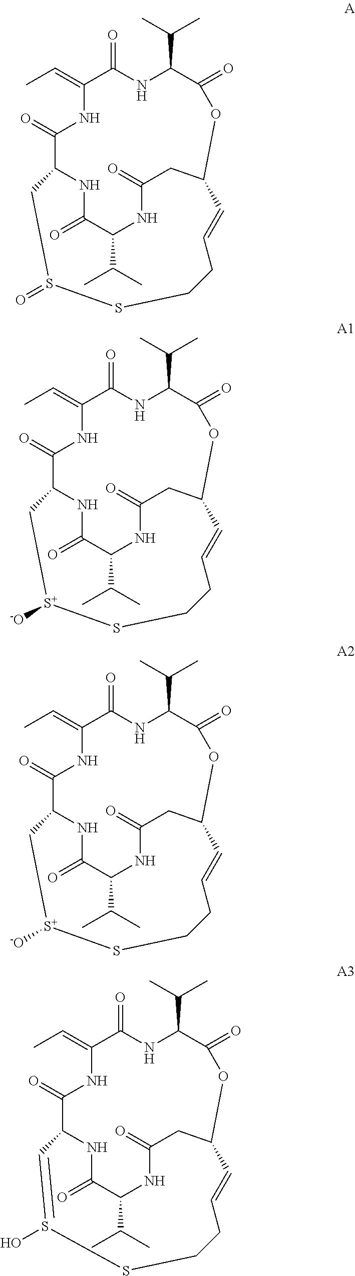

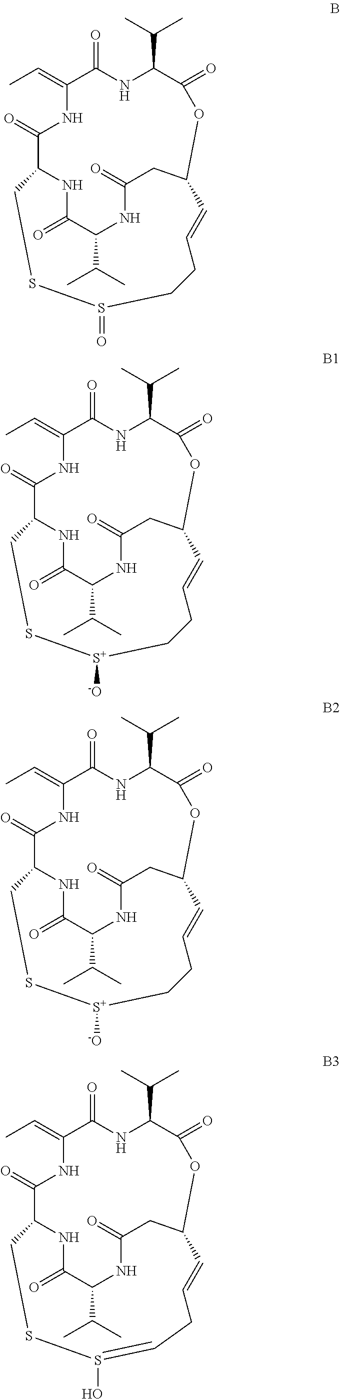

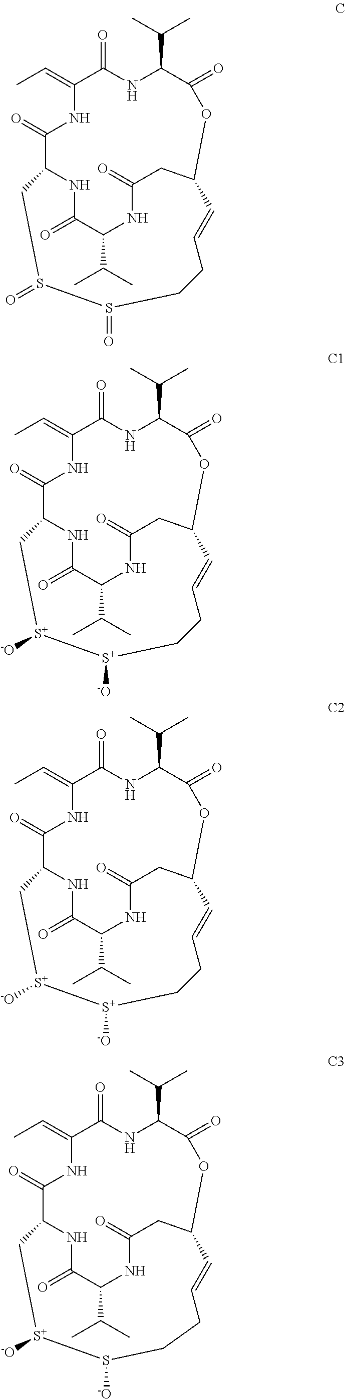

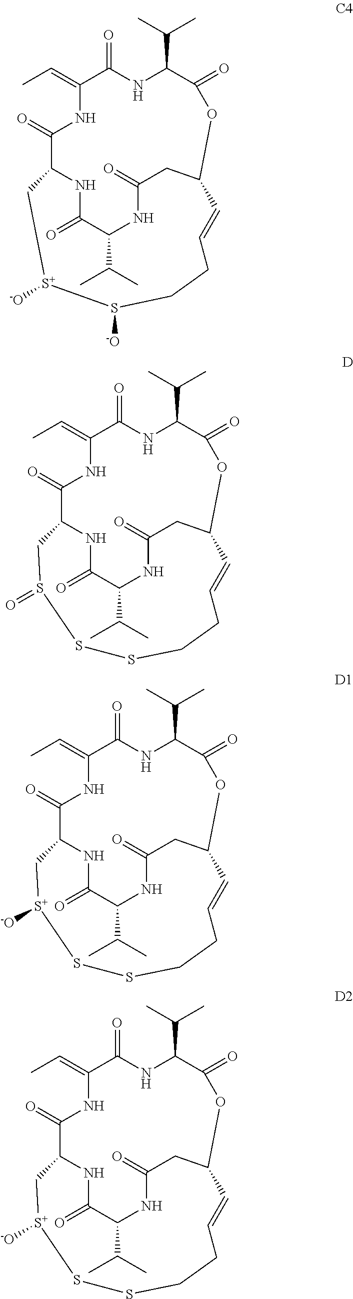

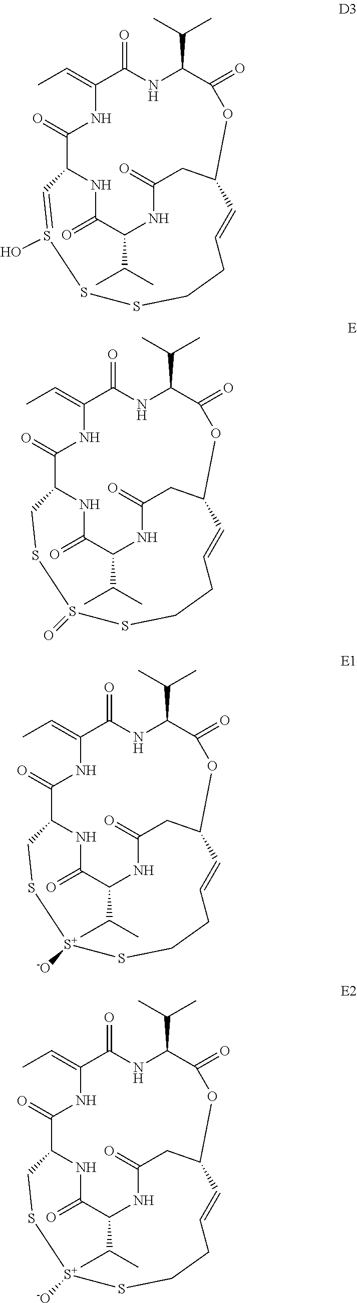

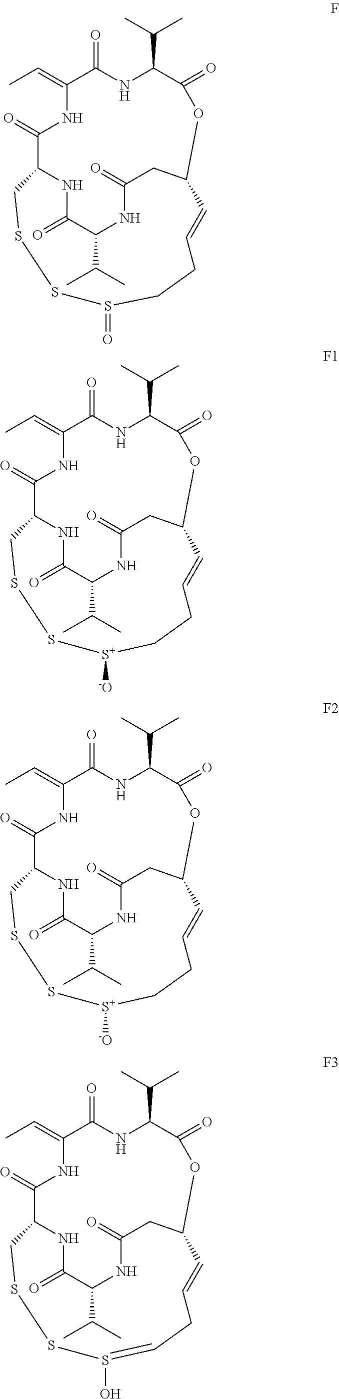

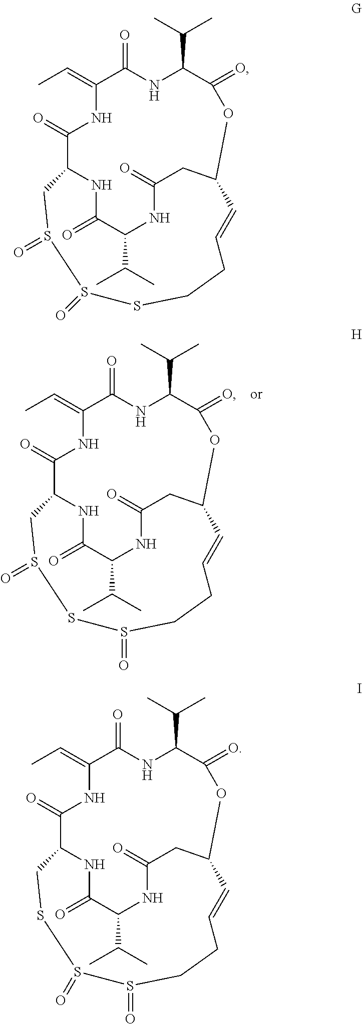

5. A method for preparing a compound represented by formula (I) as claimed in claim 1 or a pharmaceutically acceptable salt, stereoisomer, or optical isomer thereof, said method comprising: oxidizing the compound of formula (II) with an oxidant to obtain the compound of formula (I) 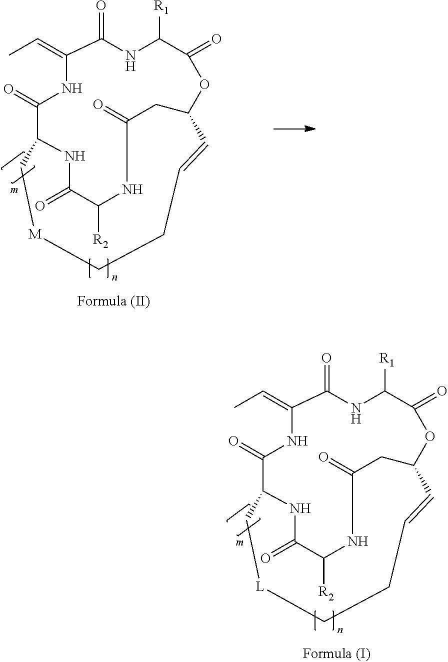
wherein, R₁, R₂, L, n and m are as defined in claim 1; M is selected from —S—, —SS—, or —SSS—.

6. The method as claimed in claim 5, wherein said oxidant is selected from any one or more of m-chloroperbenzoic acid, peracetic acid, Oxone, cumene hydroperoxide, tert-butyl hydroperoxide, acetone peroxide and hydrogen peroxide

7. A pharmaceutical composition, comprising an effective dose of the compound or a pharmaceutically acceptable salt, stereoisomer or optical isomer thereof as claimed in claim 1.

8. The pharmaceutical composition as claimed in claim 7, which is suitable for oral, rectal, parenteral, intranasal or transdermal administration or by inhalation or by suppository administration.

9. The pharmaceutical composition as claimed in claim 7, which is in the form of tablets, capsules, troches, lozenges, water or oil suspensions, dispersible powder, granules, or sublingual tablets.

10. A use of the compound as claimed in claim 1 in the preparation of a drug for the treatment of disorders resulting from cell proliferation and/or angiogenesis or disorders associated with or accompanying cell proliferation and/or angiogenesis, wherein the disorders are selected from: anti-proliferative disorders (such as cancer); neurodegenerative diseases, including: Huntington disease, polyglutamine disease, Parkinson's disease, Alzheimer's disease, epileptic seizures, striatum degeneration, progressive supranuclear palsy, insufficiency of torsional strain, spastic torticollis and dyskinesia, familial tremor, Tourette's syndrome, diffuse Lewy body disease (DLBD), Pick's disease, intracranial hemorrhage, primary lateral sclerosis, spinal muscular atrophy, amyotrophic lateral sclerosis, hypertrophic interstitial polyneuropathy, retinitis pigmentosa, hereditary optic atrophy, hereditary spastic paraplegia, progressive ataxia and Shy-Drager syndrome; metabolic diseases, including: type 2 diabetes; eye degenerative diseases, including: glaucoma, age-related macular degeneration, rubeotic glaucoma; inflammatory and/or immune system disorders, including: rheumatoid arthritis, osteoarthritis, juvenile chronic arthritis, graft versus host disease, psoriasis, asthma, spondyloarthropathies, psoriasis, Crohn's disease, inflammatory bowel disease, colonic ulcer, alcoholic hepatitis, diabetes, Sjoegren's syndrome, multiple sclerosis, ankylosing spondylitis, membranous glomerulopathy, discogenic pain, systemic lupus erythematosus; diseases involving angiogenesis, including: cancer, psoriasis, rheumatoid arthritis; psychological disorders, including: bipolar disorder, schizophrenia, mania, depression and dementia; cardiovascular diseases, including: cardiac failure, restenosis and atherosclerosis; fibrotic diseases, including: hepatic fibrosis, cystic fibrosis and vascular fibrillation; infectious diseases, including: fungal infections, such as: candida albicans, bacterial infections, viral infections, such as: herpes simplex, protozoal infections, such as: malaria, leishmania infection, Trypanosoma brucei infection, toxoplasmosis and coccidiosis; and hematopoietic disorders, including: thalassemia, anemia and sickle-cell anemia.

11. The compound as claimed in claim 2, wherein, L is selected from:

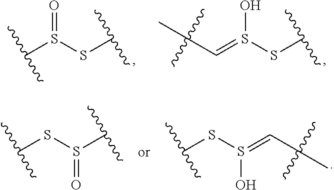 [SMILES: *SS(*)=O ; */C=S(\O)S* ; *SS(*)=O ; */C=S(\O)S*]

12. The pharmaceutical composition as claimed in claim 7, wherein R₁ and R₂ are independently selected from H, C₁-C₆ alkyl or C₁-C₆ alkoxy.

13. The pharmaceutical composition as claimed in claim 12, wherein L is selected from:

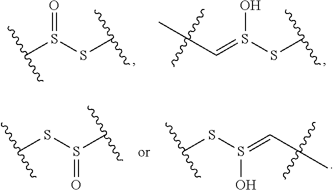 [SMILES: *SS(*)=O ; */C=S(\O)S* ; *SS(*)=O ; */C=S(\O)S*]

14. The pharmaceutical composition as claimed in claim 7, wherein L is selected from:

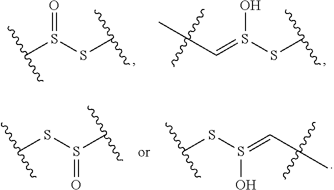 [SMILES: *SS(*)=O ; */C=S(\O)S* ; *SS(*)=O ; */C=S(\O)S*]

15. The pharmaceutical composition as claimed in claim 7, wherein said compound represented by formula (I) is selected from:

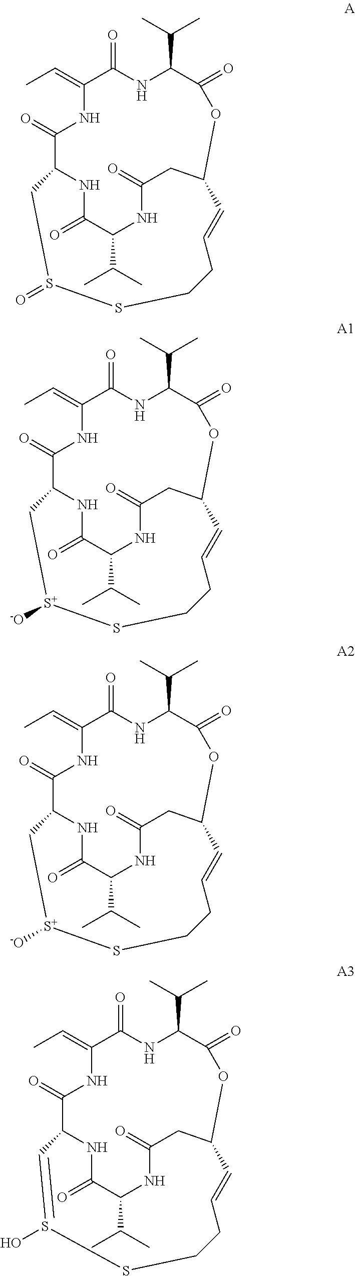

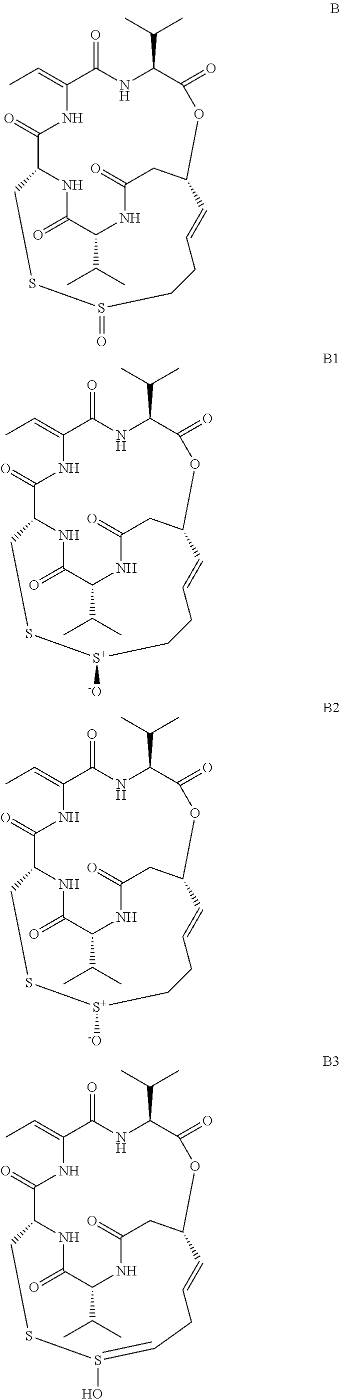

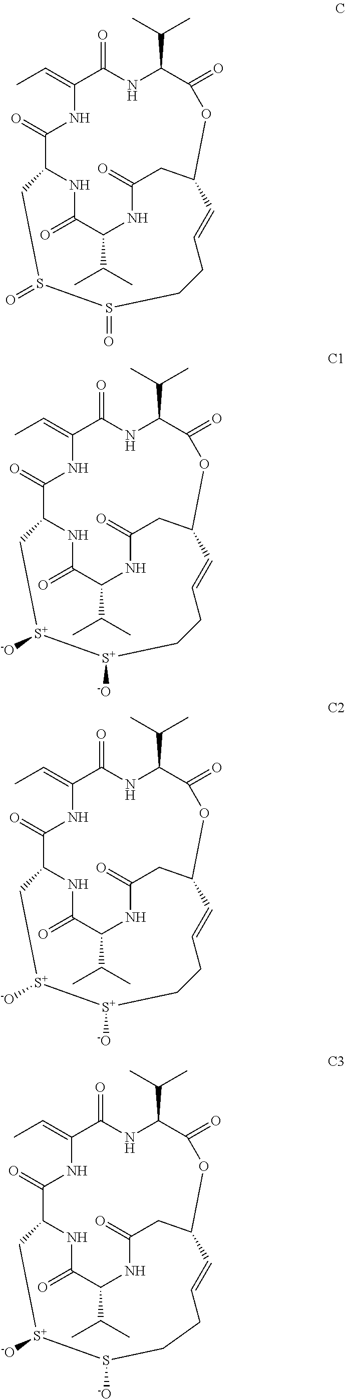

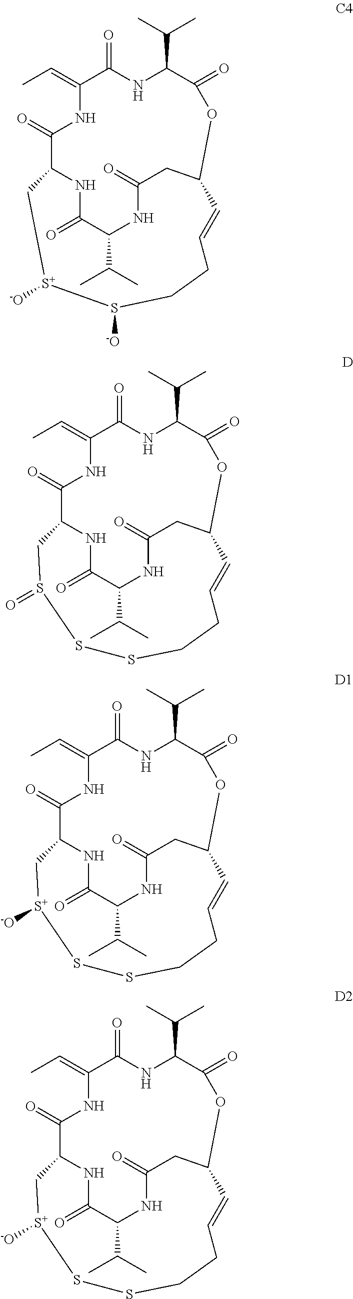

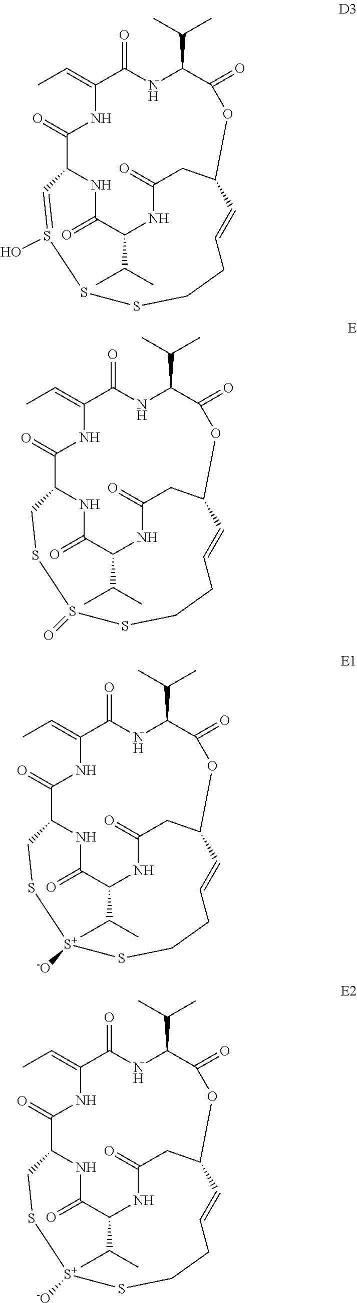

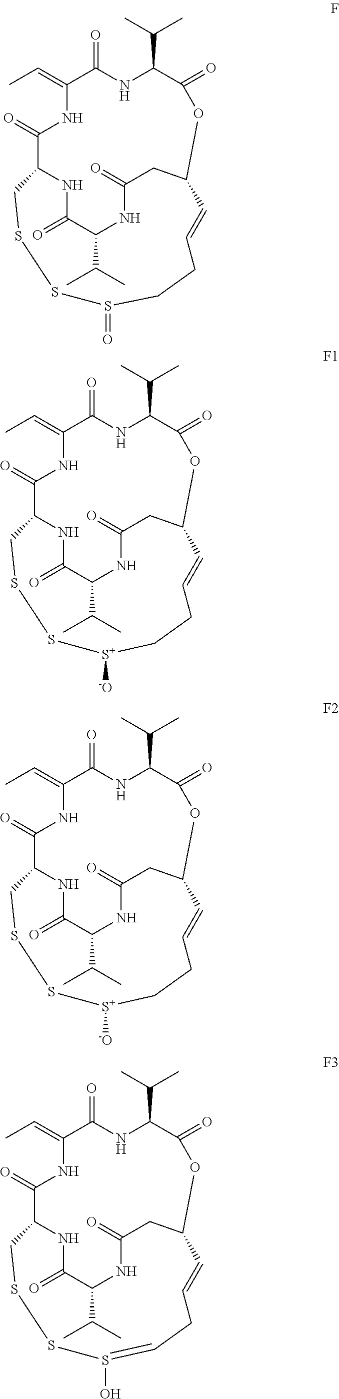

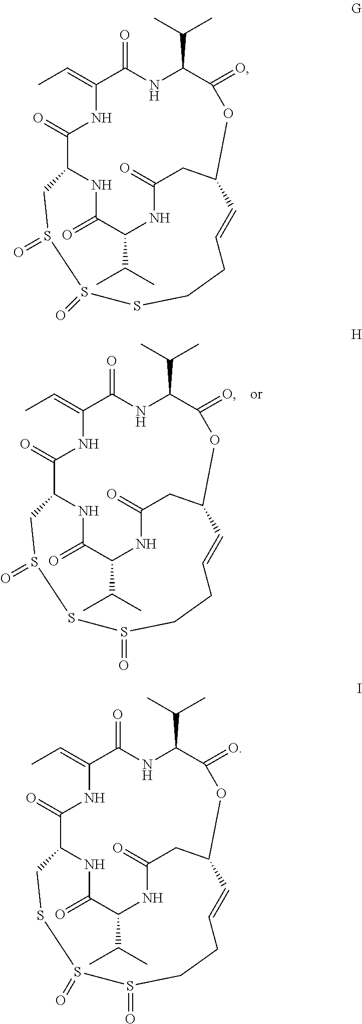

16. The use of the compound as claimed in claim 10, wherein R₁ and R₂ are independently selected from H, C₁-C₆ alkyl or C₁-C₆ alkoxy.

17. The use of the compound as claimed in claim 16, wherein L is selected from:

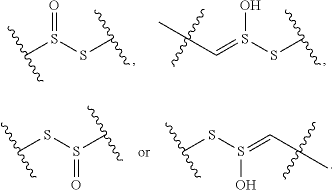 [SMILES: *SS(*)=O ; */C=S(\O)S* ; *SS(*)=O ; */C=S(\O)S*]

18. The use of the compound as claimed in claim 10, wherein L is selected from:

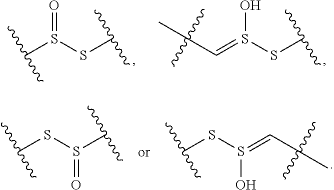 [SMILES: *SS(*)=O ; */C=S(\O)S* ; *SS(*)=O ; */C=S(\O)S*]

19. The use of the compound as claimed in claim 10, wherein said compound represented by formula (I) is selected from: 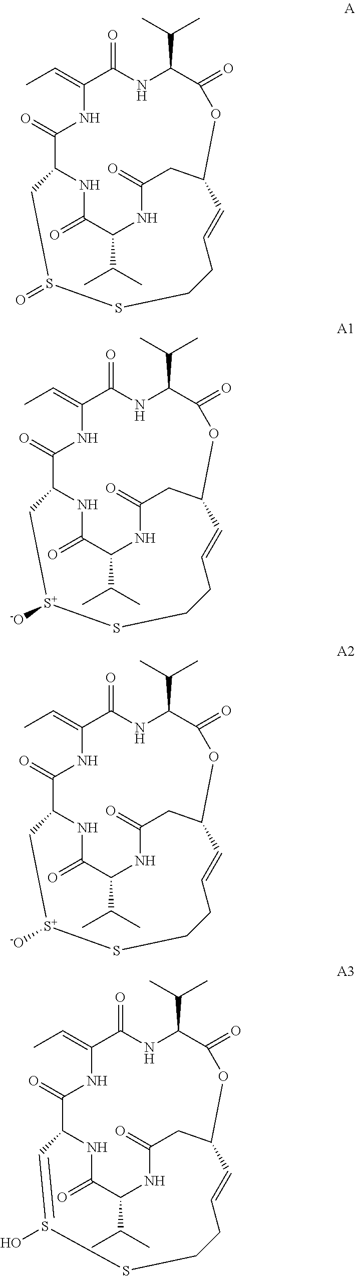 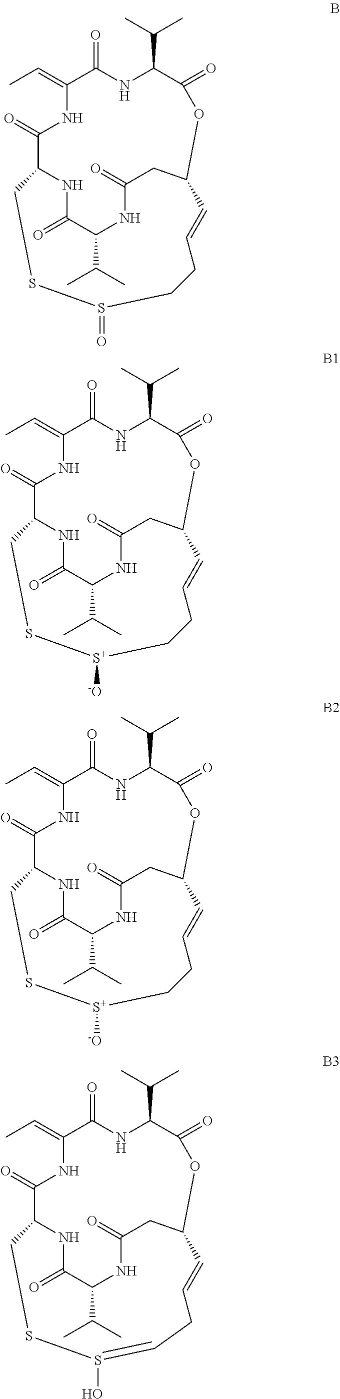 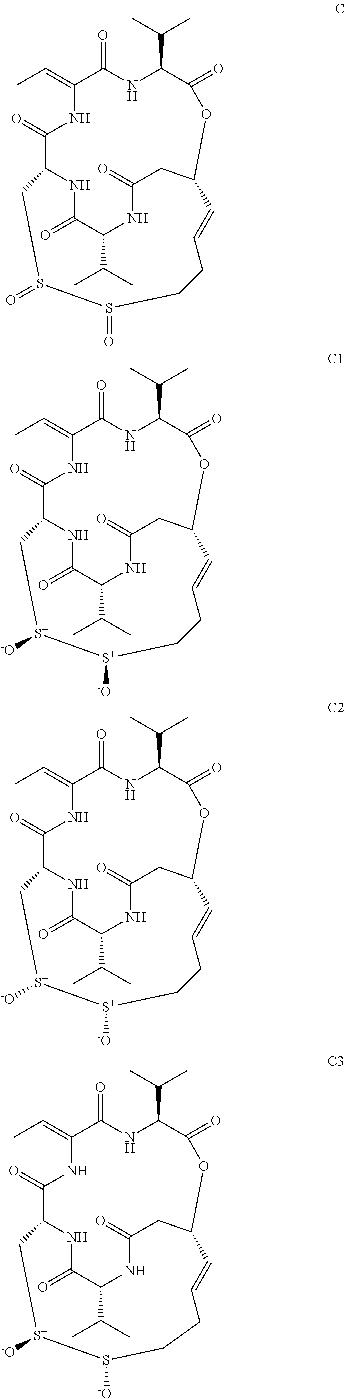 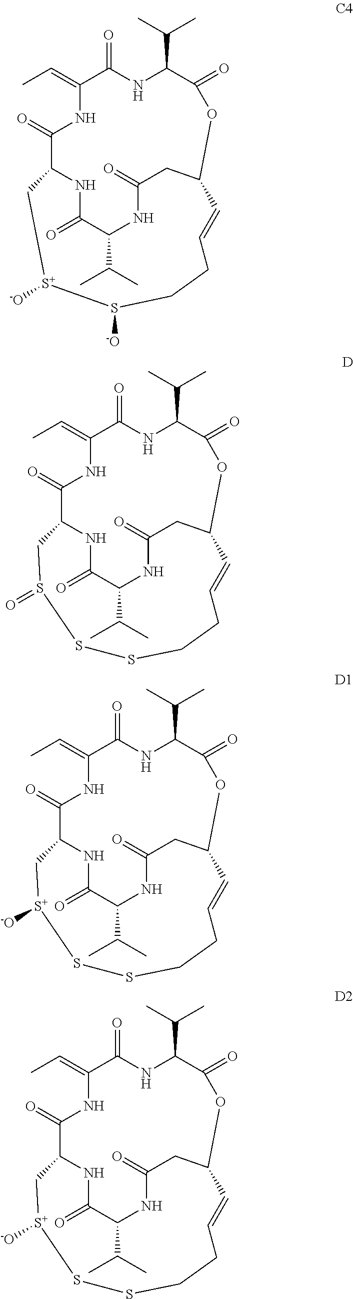 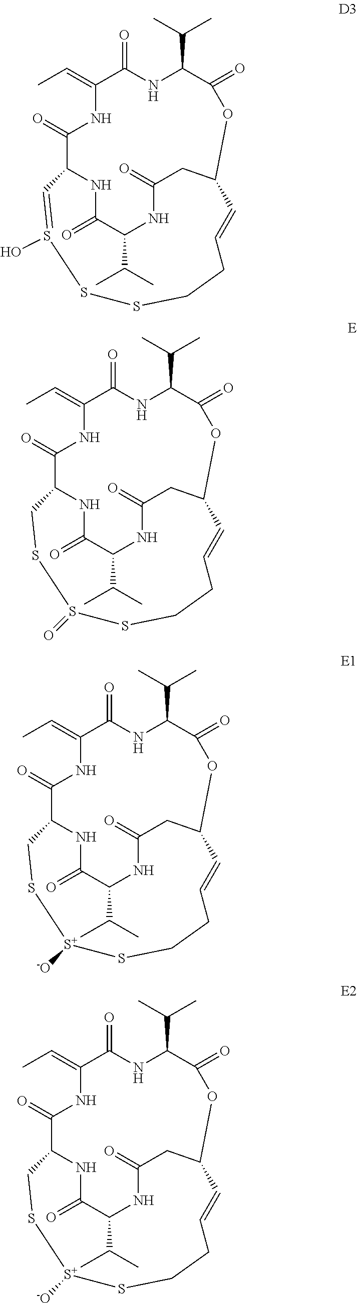 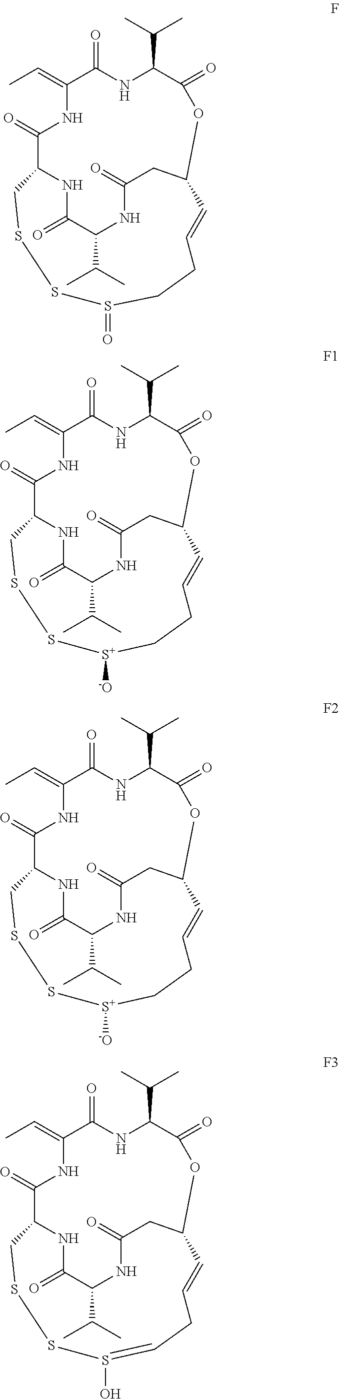 [chemical structure]
